(19) United States
(12) Patent Application Publication
(10) Pub. No.: US 2023/0181500 A1
(43) Pub. Date: Jun. 15, 2023

(54) Naloxone Pharmaceutical Formulations for Intranasal (IN) Delivery

(71) Applicant: Amphastar Pharmaceuticals, Inc., Rancho Cucamonga, CA (US)

(72) Inventors: Jack Yongfeng Zhang, Rancho Cucamonga, CA (US); Mary Zi-ping Luo, Rancho Cucamonga, CA (US); Jie Fei Ding, Rancho Cucamonga, CA (US); Aili Bo, Rancho Cucamonga, CA (US); Jiewen Zhu, Rancho Cucamonga, CA (US)

(21) Appl. No.: 17/917,415

(22) PCT Filed: May 3, 2021
(86) PCT No.: PCT/US2021/030501

§ 371 Date: Oct. 6, 2022

Related U.S. Application Data
(60) Provisional application No. 63/019,983, filed on May 4, 2020.

(51) Int. Cl. A61K 31/185; A61K 9/00; A61K 47/28

(52) U.S. Cl. CPC A61K 31/185; A61K 9/0043; A61K 47/28

(57) ABSTRACT

The present disclosure introduces intranasal (IN) pharmaceutical formulations having naloxone or an opioid antagonist as the active agent, and an absorption enhancer, such as a bile acid, or a salt thereof, for IN delivery. The bile acid, or salt thereof, enhances absorption of naloxone or an opioid antagonist into the bloodstream of a human subject.

### CROSS-REFERENCE TO RELATED APPLICATIONS

The present application claims the benefit of U.S. Provisional Application No. 63/019,983, filed on May 4, 2020, which is incorporated by reference herein in its entirety.

### FIELD

The present disclosure generally pertains to pharmaceutical formulations suitable for intranasal (IN) delivery, in which the formulations include naloxone, such as naloxone hydrochloride (HCl), or an opioid antagonist, as the active agent, and a bile acid, or a salt thereof (e.g. sodium taurocholate (STC)), as an absorption enhancer for enhancing the absorption of naloxone into a human subject's bloodstream by IN delivery.

### BACKGROUND

### Naloxone Background

Naloxone, such as naloxone hydrochloride (HCl) is an opioid antagonist that reduces or minimizes opioid effects by competing for the same receptor sites. Naloxone HCl reverses the effects of opioids, including respiratory depression, sedation, and hypotension. Naloxone HCl also reverses the psychotomimetic and dysphoric effects of agonist-antagonists such as pentazocine. Thus, naloxone HCl is indicated for the emergency treatment of known or suspected opioid overdose, as manifested by respiratory and/or central nervous system depression.

Naloxone HCl has the chemical formula C₁₉H₂₁NO₄·HCl and a molecular weight of 363.84 grams per mole (g/mol). More particularly, naloxone HCl is the hydrochloride salt of 17-allyl-4,5α-epoxy-3,14-dihydroxymorphinan-6-one. Naloxone HCl has the following chemical structure:

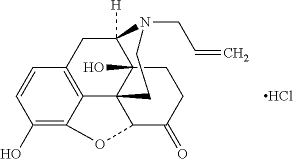 [SMILES: C=CCN1CC[C@]23c4c5ccc(O)c4O[C@H]2C(=O)CC[C@@]3(O)[C@H]1C5 ; Cl]

Naloxone HCl is currently available in a pre-filled syringe, or a pre-filled, single dose intranasal (IN) spray. For example, International Medication Systems, Ltd. (El Monte, Calif.) currently markets naloxone HCl (2 mg/2 mL) in a pre-filled syringe. Alternatively, Adapt Pharma Operations Ltd. currently markets the Narcan® IN spray, which is a pre-filled, single dose IN spray and one spray delivers 0.1 mL of the formulation, including a dose amount of 4 mg (or 2 mg) naloxone HCl. 2 mg or 4 mg naloxone HCl is equivalent to 1.8 mg or 3.6 mg naloxone, respectively.

### Practical Disadvantages of Injectable Naloxone

On a practical level, patients may be reluctant to self-inject because of a general (or extreme) fear of needles, bleeding, pain/discomfort of a needle puncture, bruising, fear of needing multiple attempts to properly self-inject, anxiety, and inability to self-inject properly in emergency situations when the patient may not be calm or composed. Repeated training may be needed for a patient to learn how to properly self-inject.

Also, naloxone HCl is often used in emergency situations where a patient experiences a drug overdose, and in such stressful situations, the patient may forget the proper techniques due to the lack of routine use. And especially in emergency situations, patients may not be in a calm or composed state of mind to properly self-inject the drug. For example, proper IM self-injection requires the patient to know and remember the optimal sites on the human body (e.g. thigh muscle) to inject in order for the drug to be absorbed relatively quickly into the bloodstream. Otherwise, if the patient injects the IM drug at non-optimal sites of the human body, then it may take a longer period of time for the drug to be absorbed into the bloodstream, which may be counter-productive as an emergency treatment. In addition, improper self-injection may lead to bleeding, bruising, swelling, numbness, tingling, lacerations, or other pain/discomfort. On the other hand, these potential issues may be reduced or eliminated in IN self-administration due to its non-invasive nature.

### Advantages of IN Administration

The intranasal (IN) route of administration is a non-invasive drug delivery method. The nasal cavity provides a readily accessible vascular bed. For medications that can reach and cross mucous membranes, the IN route is a quick and effective alternative than traditional routes of administration such as intramuscular (IM) or intravenous (IV). In particular, the nasal cavity provides direct access to the bloodstream, thereby avoiding first-pass metabolism of portal circulation, and leading to a rapid onset of drug action. Furthermore, the absorption of medication through nasal vasculature avoids gastrointestinal and hepatic destruction of medications administered orally, and therefore nasal delivery can be more bioavailable, efficient, and cost-effective than certain oral regimens. Moreover, because the nasal mucosa and its vasculature is anatomically located close to the central nervous system, IN delivery is especially suitable in situations that may require emergent or immediate access to the CNS. Practically, IN drugs also offer several important advantages, such as being easy to use, painless, easy to carry, and self-administrable without use of needles. Compared to injectables, the non-invasive IN drugs may be more acceptable to patients who are reluctant to, anxious about, or technically challenged in self-injection.

The naloxone HCl formulation without an enhancing agent (hereafter referred to as “simple IN formulation”) upon which the marketed Narcan® IN product was based, showed that one intranasal delivery of a 40 mg/mL naloxone simple IN formulation had a Tₘₐₓ of about 30 min, Cₘₐₓ of about 5.1-5.5 ng/mL, and AUC_{inf} of about 480.6-568.8 ng/mL·min. In order to increase the drug absorption amount, reduce absorption time, or improve bioavailability of naloxone or its salt, new intranasal formulations with absorption enhancers (“enhanced IN formulations”) need to be developed.

Technical Challenges Associated with Intranasal (IN) Delivery and, Using Bile Acids and Salts Thereof as Absorption Enhancers for IN Naloxone Delivery

A potential alternative to injection delivery is to deliver the drug through the intranasal route of administration, also referred to as intranasal (IN) delivery herein. The intranasal (IN) route of administration is a non-invasive drug delivery method that can provide a rapid onset of drug action. In particular, the nasal cavity provides a direct access to the bloodstream, thereby avoiding first-pass metabolism of portal circulation, and leading to a rapid onset of drug action. By contrast, IM delivery requires the patient to inject deep into the muscle at an optimal site, otherwise the drug may not be readily absorbed into the bloodstream. In addition, IN drugs offer several practical advantages over IM, such as being easy to use, painless, easy to carry, and self-administrable without use of needles.

IN delivery utilizes drug absorption through the nasal cavity and more particularly, the nasal mucosa (also known as the respiratory mucosa), which is a highly vascularized mucous membrane that lines the nasal cavity, thereby providing a large surface area. The nasal mucosa is made up of primarily two layers, an upper epithelial layer that is predominantly lipophilic, and a sub-layer known as the lamina propria. The upper epithelial layer is generally made up of epithelium, cilia, mucus (mucin), goblet cells (mucus-producing cells), and other cells. Notably, the lamina propria is highly vascularized with an extensive network of blood vessels, which can enable a drug to be rapidly absorbed into the bloodstream. However, in order to reach these blood vessels in the lamina propria, a pharmacological challenge is developing pharmaceutical formulations that would enhance the absorption of the drug through the predominantly lipophilic upper epithelial layer.

The nasal cavity is the main passageway for air into and out of the lungs. Thus, a primary function of the nasal mucosa is to serve as an immune defense against foreign agents, such as drugs, allergens, pathogens, viruses, bacteria, dirt particles, and other airborne particulates. Consequently, it is a pharmacological challenge to assist or improve drug absorption through the nasal mucosa particularly when the drug, by itself, has intrinsically low membrane permeability.

Thus, an absorption enhancer is needed to enhance the absorption of naloxone into the bloodstream via IN delivery. This increased absorption may be achieved by enhancing the absorption of naloxone HCl into the network of blood vessels in the nasal mucosa. In this regard, another pharmacological challenge of IN delivery is to minimize or reduce the absorption enhancer's local toxicity to the nasal cavity. Minimizing local toxicity to the nasal cavity is needed because the nasal mucosa provides critical functions such as humidifying the inhaled air and serving as an immune defense against foreign agents. As a result, the nasal mucosa is one of the most commonly infected tissues and inflammation of the nasal mucosa may cause a stuffy nose, headaches, mouth breathing, and other symptoms. One such absorption enhancer is a bile acid or salt thereof.

However, historically, bile acids and salts thereof have not been successfully implemented for clinical use due to various toxicity issues. In particular, it was believed that bile acids and salts thereof had limited clinical use because of the irreversible damage to the mucosa and ciliotoxicity. In addition, it was believed that bile acids and salts thereof may cause nasal irritation when used above a certain concentration, such as a concentration above 0.3% (3 mg/mL). In another study, esophageal or throat mucosa showed tolerance of up to about 250 mg/mL of bile acids and salts thereof such as STC or STCDC.

Sodium taurocholate (STC) is an example of a bile acid or salt thereof that naturally occurs as a main ingredient of the bile of carnivorous animals including humans. Physiologically, it can solubilize fats for absorption in the gut mucosa. The U.S. Food and Drug Administration (FDA) included taurocholic acid and several other bile acids on the Generally Recognized As Safe (GRAS) substance list. To date, the U.S. Food and Drug Administration (FDA) database has not listed STC as an inactive ingredient for approved drugs. Therefore, minimizing or reducing these bile acids or their salts' toxicity effects to the nasal cavity is a major technical challenge, which may be achieved by meticulously choosing the enhancer concentration and the pH of the formulation.

Accordingly, these competing pharmacological challenges require IN pharmaceutical formulations that can deliver naloxone into the bloodstream while also minimizing or reducing the absorption enhancer's toxicity. Embodiments of the present disclosure solve these pharmacological challenges by introducing IN pharmaceutical formulations including naloxone HCl, or a hydrate thereof, and a bile acid, or a salt thereof, as the absorption enhancer.

### Addressing an Unmet Need

More specifically, the present disclosure addresses the aforementioned unmet practical and technical challenges as discussed below. First, the inclusion of an absorption enhancer in the IN formulation can not only overcome the practical hurdles of the IM formulation, but also show improved absorption and bioavailability of naloxone HCl. Second, bile acid or bile salt derivatives may serve as excellent absorption enhancers for IN formulations. However, the feasibility of bile acid or salt derivatives as enhancers seems highly dependent on the physiochemical properties of both the drug and the enhancer. To achieve enhancing effect, different drugs may necessitate different concentrations of certain types of bile acids and salts thereof, e.g. STC and STCDC, as well as varying pH and other excipients in a particular formulation. Therefore, the present invention marks the first successful IN formulation taking advantage of the application of bile acid or its salt used as an enhancer for the intranasal delivery of naloxone, resulting in decreased Tₘₐₓ, which indicates accelerated absorption. Third, in some embodiments, the optimized concentration of enhancer (STC) as disclosed in this invention is essential to achieve an optimal IN bioavailability of naloxone resulting in increasing relative bioavailability with serum naloxone levels comparable to IM delivery.

### BRIEF SUMMARY

Exemplary embodiments of the present disclosure address at least the above problems and/or disadvantages and advance the art by providing at least the features described below. Additional objects, advantages, and salient features of exemplary embodiments of the present disclosure will become apparent to those skilled in the art from the following detailed description, which, taken in conjunction with the annexed drawings, discloses exemplary embodiments of the present disclosure.

Disclosed are pharmaceuticals formulations suitable for intranasal (IN) delivery comprising naloxone, or a hydrate thereof, and an absorption enhancer comprising a bile salt, and other pharmaceutically acceptable excipients. In some embodiments, the bile salt is one of a trihydroxy conjugate, a dihydroxy conjugate, or an unconjugated form. In some embodiments, the bile salt is sodium taurocholate (STC).

Therefore, in some embodiments of the pharmaceutical formulation, the naloxone, or a hydrate thereof, is present at a concentration of 5.0 mg/mL to 50.0 mg/mL, including any range subsumed therein. In some embodiments, the naloxone, or a hydrate thereof, is present in a dose amount of 0.50 mg to 20.0 mg, including any range subsumed therein. In some embodiments, the bile acid, or the salt thereof (e.g., STC), is present at a concentration of 1.0 mg/mL to 15.0 mg/mL, including any range subsumed therein. In some embodiments, the bile acid, or the salt thereof (e.g., STC), is present in a dose amount of 0.1 mg to 1.5 mg. Other exemplary embodiments of this disclosure will be detailed herein.

Also disclosed are the increased AUC and Cₘₐₓ levels of STC enhanced IN formulations, which are comparable to IM delivery, with a decreased Tₘₐₓ suggesting faster onset of drug action in animal study. Importantly, the relative bioavailability (RBA) increases as the concentration of STC increases in the IN formulation.

More specifically, the claimed and exemplary formulations in this invention have the following technical advantages over the known IM or simple IN formulations of naloxone or its salt thereof, which do not comprise an absorption enhancer present.

Technical advantages of the current invention include but are not limited to: improved absorption of naloxone or its salt thereof, improved bioavailability of naloxone or its salt thereof, increased relative bioavailability of naloxone or its salt thereof, reduced Tₘₐₓ of naloxone or its salt thereof, increased Cₘₐₓ of naloxone or its salt thereof, increased AUC of naloxone or its salt thereof, improved lipophilicity of naloxone or its salt thereof, improved membrane permeation of naloxone or its salt thereof, reduced local toxicity from enhancers or other excipients, optimized enhancer concentration, optimized pH of the formulation, optimized tonicity of the formulation, and ease of drug product application.

Specifically, in at least one embodiment (Table 1), one dose of intranasal delivery of a 40 mg/mL naloxone IN formulation enhanced with 8 mg/mL STC (Arm T3) had a reduced Tₘₐₓ of about 6.6 min, increased Cₘₐₓ of about 414 ng/mL, and increased AUC_{0-180 min} of over 20000 ng/mL·min (Table 2), and increased relative bioavailability (RBA) of about 60% (Table 3); whereas unenhanced simple IN formula (Arm T1) had a Tₘₐₓ of about 18 min Cₘₐₓ of about 162 ng/mL, AUC_{0-180 min} of about 12000 ng/mL·min (Table 2), and relative bioavailability (RBA) of less than 30% (Table 3). It is also noted that the STC-enhanced IN delivery can provide comparable naloxone nasal absorbance to IM delivery method.

Particularly, the claimed and exemplary formulations in this invention have the aforementioned technical advantages over the known IM or simple IN formulations of naloxone or its salt thereof, which do not comprise an absorption enhancer. In several preferred example formulation embodiments, the disclosed pharmaceutical formulations for intranasal (IN) delivery, comprise naloxone or its salt or a hydrate thereof, and an absorption enhancer, preferably a bile acid or salt such as STC, in the concentration range of preferably 4.0-12.0 mg/mL, further comprising a preferably 1.0-10.0 mg/mL of a tonicity agent, wherein the pharmaceutical formulation has a preferred pH of 3.0 to 5.0, wherein pH is adjusted using a pH adjustor, is advantageous over IM or simple IN delivery of naloxone or its salt of a hydrate thereof, without the presence of an enhancer.

The present disclosure provides IN pharmaceutical formulations comprising an opioid antagonist and pharmaceutically acceptable excipients.

In some embodiments, the IN pharmaceutical formulation comprises naloxone, or a hydrate thereof; and an absorption enhancer comprising a bile acid, or a salt thereof; wherein the formulation has a pH of about 3 to about 5.

In some embodiments, the naloxone, or the hydrate thereof, is present at a concentration of about 5 mg/mL to about 50 mg/mL.

In some embodiments, the naloxone hydrate is naloxone HCl dihydrate.

In some embodiments, the naloxone is naloxone HCl anhydrous.

In some embodiments, the naloxone HCl dihydrate is present in a dose amount of about 0.50 mg to about 20 mg.

In some embodiments, the naloxone HCl anhydrous is present in a dose amount of about 0.50 mg to about 20 mg.

In some embodiments, the bile acid, or the salt thereof, is present at a concentration of about 1 mg/mL to about 15 mg/mL.

In some embodiments, the bile acid, or the salt thereof, is present in a dose amount of about 0.1 mg to about 1.5 mg.

In some embodiments, the bile acid, or the salt thereof, is a trihydroxy conjugate.

In some embodiments, the bile acid, or the salt thereof, is a trihydroxy conjugate selected from the group consisting of glycocholate (GC), taurocholate (TC), glycohyocholate (GHC), taurohyocholate (THC), tauro-α-muricholate (T-α-MC), tauro-β-muricholate (T-β-MC), and combinations thereof.

In some embodiments, the bile acid, or the salt thereof, is a trihydroxy conjugate selected from the group consisting of sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), and combinations thereof. Other suitable forms of salts are possible, such as substituting sodium with potassium (e.g. potassium glycocholate).

In some embodiments, the bile acid, or the salt thereof, is a dihydroxy conjugate.

In some embodiments, the bile acid, or the salt thereof, is a dihydroxy conjugate selected from the group consisting of sodium tauroursodeoxycholate (STUDC), sodium taurohyodeoxycholate (STUDC), sodium glycohyodeoxycholate (SGUDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), and combinations thereof.

In some embodiments, the bile acid, or the salt thereof, is a dihydroxy conjugate selected from the group consisting of tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), and combinations thereof.

In some embodiments, the bile acid, or the salt thereof, is an unconjugated form.

In some embodiments, the bile acid, or the salt thereof, is an unconjugated form selected from the group consisting of cholate, deoxycholate (DC), chenodeoxycholate (CDC), and combinations thereof.

In some embodiments, the bile acid, or the salt thereof, is an unconjugated form selected from the group consisting of sodium cholate (SC), sodium deoxycholate (SDC), sodium chenodeoxycholate (SCDC), and combinations thereof.

In some embodiments, the bile acid, or the salt thereof, is STC.

In some embodiments, the STC is present at a concentration of about 1 mg/mL to about 15 mg/mL.

In some embodiments, the bile acid, or the salt thereof, is STCDC.

In some embodiments, the formulation is an aqueous solution having a dose volume of about 0.05 mL to about 0.5 mL.

In some embodiments, the formulation further comprises a tonicity agent.

In some embodiments, the tonicity agent has a concentration of about 1 mg/mL to about 10 mg/mL.

In some embodiments, the tonicity agent is selected from the group consisting of sodium chloride, dextrose, glucose, glycerin, cellulose, mannitol, polysorbate, propylene glycol, sodium iodide, and a combination thereof.

In some embodiments, the pH is about 3 to about 4.

In some embodiments, the pH is about 3.6.

In some embodiments, the formulation comprises a pH adjustor.

In some embodiments, the pH adjustor is hydrochloric acid.

In some embodiments, the IN pharmaceutical formulation comprises about 30 mg/mL to about 50 mg/mL of naloxone, or a hydrate thereof; about 4 mg/mL to about 12 mg/mL of an absorption enhancer comprising STC; about 1 mg/mL to about 10 mg/mL of a tonicity agent; and a dose volume of about 0.1 mL to about 0.25 mL; wherein the IN pharmaceutical formulation has a pH of about 3 to about 5.

In some embodiments, the IN pharmaceutical formulation consists of about 30 mg/mL to about 50 mg/mL of naloxone, or a hydrate thereof; about 4 mg/mL to about 12 mg/mL of an absorption enhancer comprising STC; about 1 mg/mL to about 10 mg/mL of a tonicity agent; and a dose volume of about 0.1 mL to about 0.25 mL; wherein the IN pharmaceutical formulation has a pH of about 3 to about 5.

In some embodiments, the IN pharmaceutical formulation consists essentially of about 30 mg/mL to about 50 mg/mL of naloxone, or a hydrate thereof; about 4 mg/mL to about 12 mg/mL of an absorption enhancer comprising STC; about 1 mg/mL to about 10 mg/mL of a tonicity agent; and a dose volume of about 0.1 mL to about 0.25 mL; wherein the IN pharmaceutical formulation has a pH of about 3 to about 5.

In some embodiments, the IN pharmaceutical formulation comprises about 35 mg/mL to about 50 mg/mL of naloxone, or a hydrate thereof; about 4 mg/mL to about 1 mg/mL of an absorption enhancer comprising STC; about 1 mg/mL to about 10 mg/mL of a tonicity agent; a dose volume of about 0.25 mL; pH adjustor as needed; and water; wherein the IN pharmaceutical formulation has a pH of about 3 to about 5.

In some embodiments, the IN pharmaceutical formulation comprises an opioid antagonist, or a hydrate thereof; and an absorption enhancer comprising a bile acid, or a salt thereof; wherein the IN pharmaceutical formulation has a pH of about 3 to about 5; and wherein the IN pharmaceutical formulation is for IN delivery.

In some embodiments, the opioid antagonist is selected from the group consisting of naloxone, naltrexone, nalmefene, diprenorphine, nalorphine, nalorphine dinicotinate, levallorphan, samidorphan, nalodeine, and combinations thereof.

In some embodiments, the opioid antagonist, or the hydrate thereof, is present at a concentration of about 5 mg/mL to about 50 mg/mL.

In some embodiments, the bile acid, or the salt thereof, is present at a concentration of about 1 mg/mL to about 15 mg/mL.

In some embodiments, the bile acid, or the salt thereof, is present in a dose amount of about 0.1 mg to about 1.5 mg.

In some embodiments, the bile acid, or the salt thereof, is a trihydroxy conjugate.

In some embodiments, the bile acid, or the salt thereof, is a trihydroxy conjugate selected from the group consisting of glycocholate (GC), taurocholate (TC), glycohyocholate (GHC), taurohyocholate (THC), tauro-α-muricholate (T-α-MC), tauro-β-muricholate (T-β-MC), and combinations thereof.

In some embodiments, the bile acid, or the salt thereof, is a trihydroxy conjugate selected from the group consisting of sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), and combinations thereof. Other suitable forms of salts are possible, such as substituting sodium with potassium (e.g. potassium glycocholate).

In some embodiments, the bile acid, or the salt thereof, is a dihydroxy conjugate.

In some embodiments, the bile acid, or the salt thereof, is a dihydroxy conjugate selected from the group consisting of sodium tauroursodeoxycholate (STUDC), sodium taurohyodeoxycholate (STUDC), sodium glycohyodeoxycholate (SGUDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), and combinations thereof.

In some embodiments, the bile acid, or the salt thereof, is a dihydroxy conjugate selected from the group consisting of tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), and combinations thereof.

In some embodiments, the bile acid, or the salt thereof, is an unconjugated form.

In some embodiments, the bile acid, or the salt thereof is an unconjugated form selected from the group consisting of cholate, deoxycholate (DC), chenodeoxycholate (CDC), and combinations thereof.

In some embodiments, the bile acid or salt thereof is an unconjugated form selected from the group consisting of sodium cholate (SC), sodium deoxycholate (SDC), sodium chenodeoxycholate (SCDC), and combinations thereof.

In some embodiments, the bile acid, or the salt thereof, is STC.

In some embodiments, the STC is present at a concentration of about 1 mg/mL to about 15 mg/mL.

In some embodiments, the bile acid, or the salt thereof, is STCDC.

In some embodiments, the pharmaceutical formulation is in an aqueous solution having a dose volume of about 0.05 mL to about 0.5 mL.

In some embodiments, the formulation further comprises a tonicity agent.

In some embodiments, the tonicity agent has a concentration of about 1 mg/mL to about 10 mg/mL.

In some embodiments, the tonicity agent is selected from the group consisting of sodium chloride, dextrose, glucose, glycerin, cellulose, mannitol, polysorbate, propylene glycol, sodium iodide, and combinations thereof.

In some embodiments, the pH is about 3 to about 4.

In some embodiments, the pH is about 3.6.

In some embodiments, the formulation further comprises a pH adjustor.

In some embodiments, the pH adjustor is hydrochloric acid.

In some embodiments, the IN pharmaceutical formulation comprises about 30 mg/mL to about 50 mg/mL of an opioid antagonist, or a hydrate thereof; about 4 mg/mL to about 12 mg/mL of an absorption enhancer comprising STC; about 1 mg/mL to about 10 mg/mL of a tonicity agent; and a dose volume of about 0.1 mL to about 0.25 mL; wherein the IN pharmaceutical formulation has a pH of about 3 to about 5.

In some embodiments, the IN pharmaceutical formulation comprises about 35 mg/mL to about 50 mg/mL of an opioid antagonist, or a hydrate thereof; about 4 mg/mL to about 12 mg/mL of an absorption enhancer comprising STC; about 1 mg/mL to about 10 mg/mL of a tonicity agent; a dose volume of about 0.25 mL; pH adjustor as needed; and water; wherein the IN pharmaceutical formulation has a pH of about 3 to about 5.

In some embodiments, the IN pharmaceutical formulation comprises an opioid antagonist, or a hydrate thereof; buprenorphine, or a pharmaceutically acceptable salt thereof; and an absorption enhancer comprising a bile acid, or a salt thereof; wherein the IN pharmaceutical formulation has a pH of about 3 to about 5.

In some embodiments, the opioid antagonist, or the hydrate thereof, is present at a concentration of about 5 mg/mL to about 50 mg/mL.

In some embodiments, the opioid antagonist is selected from the group consisting of naloxone, naltrexone, nalmefene, diprenorphine, nalorphine, nalorphine dinicotinate, levallorphan, samidorphan, nalodeine, and combinations thereof.

In some embodiments, the buprenorphine is present at a concentration of about 5 mg/mL to about 50 mg/mL.

In some embodiments, the buprenorphine is present in a dose amount of about 0.5 mg to about 20 mg.

In some embodiments, the bile acid, or the salt thereof, is present at a concentration of about 1 mg/mL to about 15 mg/mL.

In some embodiments, the bile acid, or the salt thereof, is present in a dose amount of about 0.1 mg to about 1.5 mg.

In some embodiments, the bile acid, or the salt thereof, is a trihydroxy conjugate.

In some embodiments, the bile acid, or the salt thereof, is a trihydroxy conjugate selected from the group consisting of glycocholate (GC), taurocholate (TC), glycohyocholate (GHC), taurohyocholate (THC), tauro-α-muricholate (T-α-MC), tauro-β-muricholate (T-β-MC), and combinations thereof.

In some embodiments, the bile acid, or the salt thereof, is a trihydroxy conjugate selected from the group consisting of sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), and combinations thereof. Other suitable forms of salts are possible, such as substituting sodium with potassium (e.g. potassium glycocholate).

In some embodiments, the bile acid, or the salt thereof, is a dihydroxy conjugate.

In some embodiments, the bile acid, or the salt thereof, is a dihydroxy conjugate selected from the group consisting of sodium tauroursodeoxycholate (STUDC), sodium taurohyodeoxycholate (STUDC), sodium glycohyodeoxycholate (SGUDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), and combinations thereof.

In some embodiments, the bile acid, or the salt thereof, is a dihydroxy conjugate selected from the group consisting of tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), and combinations thereof.

In some embodiments, the bile acid, or the salt thereof, is an unconjugated form.

In some embodiments, the bile acid is an unconjugated form selected from the group consisting of cholate, deoxycholate (DC), chenodeoxycholate (CDC), and combinations thereof.

In some embodiments, the bile acid, or the salt thereof, is an unconjugated form selected from the group consisting of sodium cholate (SC), sodium deoxycholate (SDC), sodium chenodeoxycholate (SCDC), and combinations thereof.

In some embodiments, the bile acid, or the salt thereof, is STC.

In some embodiments, the STC is present at a concentration of about 1 mg/mL to about 15 mg/mL.

In some embodiments, the bile acid, or the salt thereof, is STCDC.

In some embodiments, the pharmaceutical formulation is in an aqueous solution having a dose volume of about 0.05 mL to about 0.5 mL.

In some embodiments, the formulation further comprises a tonicity agent.

In some embodiments, the tonicity agent has a concentration of about 1 mg/mL to about 10 mg/mL.

In some embodiments, the tonicity agent is selected from the group consisting of sodium chloride, dextrose, glucose, glycerin, cellulose, mannitol, polysorbate, propylene glycol, sodium iodide, and combinations thereof.

In some embodiments, the pH is about 3 to about 4.

In some embodiments, the pH is about 3.6.

In some embodiments, the formulation further comprises a pH adjustor.

In some embodiments, the pH adjustor is hydrochloric acid.

In some embodiments, the IN pharmaceutical formulation comprises about 30 mg/mL to about 50 mg/mL of naloxone, or a hydrate thereof; about 30 mg/ml to about 50 mg/mL of buprenorphine, or a pharmaceutically acceptable salt thereof; about 4 mg/mL to about 12 mg/mL of an absorption enhancer comprising STC; about 1 mg/mL to about 10 mg/mL of a tonicity agent; and a dose volume of about 0.1 mL to about 0.25 mL; wherein the IN pharmaceutical formulation has a pH of about 3 to about 5.

In some embodiments, the IN pharmaceutical formulation comprises about 35 mg/mL to about 50 mg/mL of naloxone, or a hydrate thereof; about 35 mg/mL to about 50 mg/mL of buprenorphine, or a pharmaceutically acceptable salt thereof; about 4 mg/mL to about 12 mg/mL of an absorption enhancer comprising STC; about 1 mg/mL to about 10 mg/mL of a tonicity agent; a dose volume of about 0.25 mL; pH adjustor as needed; and water; wherein the IN pharmaceutical formulation has a pH of about 3 to about 5.

In some embodiments, the formulation comprises an additional absorption enhancer.

In some embodiments, the absorption enhancer is selected from the group consisting of dodecyl maltoside, benzalkonium chloride, oleic acid, polysorbate 20, polysorbate 80, and sodium lauryl sulfate.

In some embodiments, the additional absorption enhancer is present at a concentration of about 0.001 mg/mL.

In some embodiments, the additional absorption enhancer is present at a concentration of about 0.01 mg/mL.

In some embodiments, the additional absorption enhancer is present at a concentration of about 0.1 mg/mL.

In some embodiments, the additional absorption enhancer is present at a concentration of about 1 mg/mL.

In some embodiments, the additional absorption enhancer is present at a concentration of about 2.5 mg/mL.

In some embodiments, the formulation comprises a preservative.

In some embodiments, the preservative is selected from the group consisting of benzalkonium chloride, cyclodextrins, fusidic acid derivatives, phosphatidylcholines, and microspheres and liposomes.

In some embodiments, the preservative is present at a concentration of about 0.001 mg/mL.

In some embodiments, the additional absorption enhancer is present at a concentration of about 0.01 mg/mL.

In some embodiments, the preservative is present at a concentration of about 0.1 mg/mL.

In some embodiments, the preservative is present at a concentration of about 1 mg/mL.

In some embodiments, the preservative is present at a concentration of about 2.5 mg/mL.

In some embodiments, the IN pharmaceutical formulation comprises an opioid antagonist, or a hydrate thereof; and a bile acid, or a salt thereof; wherein the pharmaceutical formulation has a pH of about 3 to about 5; and wherein in the IN pharmaceutical formulation is for IN delivery.

In some embodiments, the IN pharmaceutical formulation consists essentially of an opioid antagonist, or a hydrate thereof; and a bile acid, or a salt thereof; wherein the pharmaceutical formulation has a pH of about 3 to about 5; and wherein in the IN pharmaceutical formulation is for IN delivery.

In some embodiments, the IN pharmaceutical formulation consists of an opioid antagonist, or a hydrate thereof; and a bile acid, or a salt thereof; wherein the pharmaceutical formulation has a pH of about 3 to about 5; and wherein in the IN pharmaceutical formulation is for IN delivery.

In some embodiments, the IN pharmaceutical formulation for IN delivery comprises about 40 mg/mL naloxone, or a hydrate thereof; about 6 mg/mL STC; and about 2 mg/mL NaCl; wherein the IN pharmaceutical formulation has a pH of about 4.5.

In some embodiments, the IN pharmaceutical formulation for IN delivery consists essentially of about 40 mg/mL naloxone, or a hydrate thereof; about 6 mg/mL STC; and about 2 mg/mL NaCl; wherein the IN pharmaceutical formulation has a pH of about 4.5.

In some embodiments, the IN pharmaceutical formulation for IN delivery consists of about 40 mg/mL naloxone, or a hydrate thereof; about 6 mg/mL STC; and about 2 mg/mL NaCl; wherein the IN pharmaceutical formulation has a pH of about 4.5.

The present disclosure also provides methods for treating opioid overdose using IN pharmaceutical formulations comprising opioid antagonists.

In some embodiments, the method comprises delivering an about 10 μL to about 300 μL spray of an IN pharmaceutical formulation into a nostril of a subject in need thereof; wherein the IN pharmaceutical formulation comprises from about 30 mg/mL to about 50 mg/mL of an opioid antagonist, or a hydrate thereof; from about 4 mg/mL to about 12 mg/mL of an absorption enhancer comprising a bile acid, or a salt thereof; and from about 1 mg/mL to about 10 mg/mL of a tonicity agent; and wherein the IN pharmaceutical formulation has a pH of 3 to 5.

In some embodiments, the opioid antagonist is selected from the group consisting of naloxone, naltrexone, nalmefene, diprenorphine, nalorphine, nalorphine dinicotinate, levallorphan, samidorphan, and nalodeine.

In some embodiments, the IN pharmaceutical formulation further comprises a second active agent, wherein the second active agent is buprenorphine or a pharmaceutically acceptable salt thereof.

In some embodiments, the buprenorphine is present at a concentration of about 5 mg/mL to about 50 mg/mL.

In some embodiments, the opioid antagonist, or the hydrate thereof, is present at a concentration of about 5 mg/mL to about 50 mg/mL.

In some embodiments, the bile acid, or the salt thereof, is present at a concentration of about 1 mg/mL to about 15 mg/mL.

In some embodiments, the bile acid, or the salt thereof, is present in a dose amount of about 0.1 mg to about 1.5 mg.

In some embodiments, the bile acid, or the salt thereof, is a trihydroxy conjugate.

In some embodiments, the bile acid, or the salt thereof, is a trihydroxy conjugate selected from the group consisting of glycocholate (GC), taurocholate (TC), glycohyocholate (GHC), taurohyocholate (THC), tauro-α-muricholate (T-α-MC), tauro-β-muricholate (T-β-MC), and combinations thereof.

In some embodiments, the bile acid, or the salt thereof, is a trihydroxy conjugate selected from the group consisting of sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), and combinations thereof. Other suitable forms of salts are possible, such as substituting sodium with potassium (e.g. potassium glycocholate).

In some embodiments, the bile acid, or the salt thereof, is a dihydroxy conjugate.

In some embodiments, the bile acid, or the salt thereof, is a dihydroxy conjugate selected from the group consisting of sodium tauroursodeoxycholate (STUDC), sodium taurohyodeoxycholate (STUDC), sodium glycohyodeoxycholate (SGUDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), and combinations thereof.

In some embodiments, the bile acid, or the salt thereof, is a dihydroxy conjugate selected from the group consisting of tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), and combinations thereof.

In some embodiments, the bile acid, or the salt thereof, is an unconjugated form.

In some embodiments, the bile acid, or the salt thereof, is an unconjugated form selected from the group consisting of cholate, deoxycholate (DC), chenodeoxycholate (CDC), and combinations thereof.

In some embodiments, the bile acid, or the salt thereof, is an unconjugated form selected from the group consisting of sodium cholate (SC), sodium deoxycholate (SDC), sodium chenodeoxycholate (SCDC), and combinations thereof.

In some embodiments, the bile acid, or the salt thereof, is STC.

In some embodiments, the bile acid, or the salt thereof, is STC present at a concentration of about 1 mg/mL to about 15 mg/mL.

In some embodiments, the bile acid, or the salt thereof, is STCDC.

In some embodiments, the IN pharmaceutical formulation is in an aqueous solution having a dose volume of about 0.05 mL to about 0.5 mL.

In some embodiments, the formulation further comprises a tonicity agent.

In some embodiments, the tonicity agent has a concentration of about 1 mg/mL to about 10 mg/mL.

In some embodiments, the tonicity agent is selected from the group consisting of sodium chloride, dextrose, glucose, glycerin, cellulose, mannitol, polysorbate, propylene glycol, sodium iodide, and a combination thereof.

In some embodiments, the pH is about 3 to about 4.

In some embodiments, the pH is about 3.6.

In some embodiments, the formulation further comprises a pH adjustor.

In some embodiments, the pH adjustor is hydrochloric acid.

In some embodiments, the subject is an opioid overdose patient or a suspected opioid overdose patient.

In some embodiments, the subject exhibits one or more symptoms selected from the group consisting of respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing, erratic or stopped pulse, choking or gurgling sounds, blue or purple fingernails or lips, slack or limp muscle tone, contracted pupils, and vomiting.

In some embodiments, the subject exhibits respiratory depression or cardiovascular depression.

In some embodiments, the respiratory depression is caused by the illicit use of opioids, or by an accidental misuse of opioids.

In some embodiments, the subject is free from respiratory depression for at least about 2 hours following treatment comprising delivery of the therapeutically effective amount of the opioid antagonist.

In some embodiments, the subject is free from respiratory depression for at least about 6 hours following treatment comprising delivery of the therapeutically effective amount of the opioid antagonist.

In some embodiments, the time to peak (Tₘₐₓ) is less than about 15 minutes.

In some embodiments, the time to peak (Tₘₐₓ) is less than about 10 minutes.

In some embodiments, the time to peak (Tₘₐₓ) is less than about 7 min.

In some embodiments, the serum drug level is comparable to the serum drug level obtained by intramuscular (IM) delivery.

In some embodiments, the peak serum concentration of the opioid antagonist (Cₘₐₓ) is from about 200 ng/mL to about 500 ng/mL.

In some embodiments, the Cₘₐₓ is from about 400 ng/mL to about 1000 ng/mL.

In some embodiments, the 30 min area under the curve (AUC_{30 min}) is from about 4500 ng/mL·min to about 7000 ng/mL·min.

In some embodiments, the 30 min area under the curve (AUC_{30 min}) is from about 7000 ng/mL·min to about 9000 ng/mL·min.

In some embodiments, the relative bioavailability (RBA) of the opioid antagonist is from about 40% to about 90%%.

In some embodiments, the RBA of the opioid antagonist is at least about 1.5 times greater than the RBA in the absence of an absorption enhancer.

In some embodiments, the RBA of the opioid antagonist is from about 60% to about 100%.

In some embodiments, the RBA of the opioid antagonist is at least about 2 times greater than the RBA in the absence of an absorption enhancer.

### BRIEF DESCRIPTION OF THE DRAWINGS

The above and other exemplary features and advantages of certain exemplary embodiments of the present disclosure will become more apparent from the following description of certain exemplary embodiments thereof when taken in conjunction with the accompanying drawings in which:

FIG. 1A is a graph illustrating the mean naloxone concentration in rat serum from 0 min. to 180 minutes utilizing STC as the bile acid, or salt thereof, which is further detailed in Example 1.

FIG. 1B is a graph illustrating the mean naloxone concentration in rat serum from 0 min. to 30 minutes utilizing STC as the bile acid, or salt thereof, which is further detailed in Example 1.

FIG. 1C is a graph illustrating the mean naloxone concentration in rat serum by IN delivery utilizing STC as the bile acid, or salt thereof, which is further detailed in Example 1.

Throughout the drawings, like reference numerals will be understood to refer to like elements, features and structures.

### DETAILED DESCRIPTION

The examples of the present disclosure are provided to assist in a comprehensive understanding of exemplary embodiments of the invention with reference to the accompanying drawing figures. While the present disclosure has been described in connection with certain embodiments, it is to be understood that the invention is not limited to the disclosed embodiments, but, on the contrary, is intended to cover various modifications and equivalent arrangements included within the spirit and scope of the appended claims, and equivalents thereof. Accordingly, those of ordinary skill in the art will recognize that various changes and modifications of the exemplary embodiments described herein can be made without departing from the scope and spirit of the disclosure.

The word “exemplary” is used herein to mean “serving as an example, instance, or illustration.” Any embodiment described herein as “exemplary” is not necessarily to be construed as preferred or advantageous over other embodiments. Likewise, the term “embodiments” does not require that all embodiments include the discussed feature, advantage or mode of operation.

Unless otherwise defined herein, scientific and technical terms used in connection with embodiments of present disclosure shall have the meanings that are commonly understood by those of ordinary skill in the art. Nomenclatures used in connection with, and techniques described herein are those known and commonly used in the art. Also, descriptions of well-known functions and constructions are omitted for clarity and conciseness.

As used herein, the singular forms “a,” “an,” and “the” are intended to include the plural forms as well, unless the context clearly indicates otherwise. It will be further understood that the terms “comprises,” “comprising,” “includes,” “including,” “have” and/or “having” when used herein, specify the presence of stated features, integers, steps, operations, elements, and/or components, but do not preclude the presence or addition of one or more other features, integers, steps, operations, elements, components, and/or groups thereof.

### IN Naloxone Pharmaceutical Formulations

Disclosed herein are pharmaceutical formulations having naloxone, or a hydrate thereof, as the active agent for IN delivery. In some embodiments, the active agent is an opioid antagonist. In some embodiments, the naloxone hydrate is naloxone HCl dihydrate. In other embodiments, the naloxone is naloxone HCl anhydrous. Naloxone HCl anhydrous has a molecular weight of 363.84 g/mol, while naloxone HCl dihydrate has a molecular weight of 399.87 g/mol. Thus, 1.0 mg of naloxone HCl anhydrous is equivalent to 1.1 mg of naloxone HCl dihydrate.

“Pharmaceutical formulation” refers to a formulation comprising at least one active agent. For brevity herein, “naloxone pharmaceutical formulations” or “naloxone formulations” each refers to pharmaceutical formulations including at least naloxone, or a hydrate thereof. In some embodiments, “pharmaceutical formulation” refers to a formulation comprising an opioid antagonist.

The term “naloxone” refers to naloxone or a pharmaceutically acceptable salt thereof. An example of a pharmaceutically acceptable salt of naloxone is naloxone HCl. Thus, in some embodiments of the pharmaceutical formulation, the naloxone is naloxone HCl. In some embodiments, the naloxone HCl is naloxone HCl anhydrous.

Opioid antagonists are compounds that block one or more opioid receptors. Non-limiting examples of opioid antagonists include naloxone, naltrexone, nalmefene, diprenorphine, nalorphine, nalorphine dinicotinate, levallorphan, samidorphan, and nalodeine, hydrates thereof, or pharmaceutically acceptable salts thereof.

The term “hydrate,” as used in the context of naloxone, refers to naloxone or a salt thereof that further includes a stoichiometric or non-stoichiometric amount of water bound by non-covalent intermolecular forces. An example of a naloxone hydrate is naloxone HCl dihydrate.

In some embodiments, the IN pharmaceutical formulation comprises naloxone, or a hydrate thereof, present in an amount of about 5 mg/mL to about 50 mg/mL or any amount range subsumed therein, including but not limited to, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL. In some aspects, the IN pharmaceutical formulation comprises naloxone, or a hydrate thereof, present in an amount of about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, or about 4 mg/mL to about 5 mg/mL.

In a preferred embodiment, the IN pharmaceutical formulation comprises naloxone, or a hydrate thereof, present in an amount of about 30 mg/mL to about 50 mg/mL.

In some embodiments, the IN pharmaceutical formulation comprises naloxone HCl dihydrate present in an amount of about 5 mg/mL to about 50 mg/mL or any amount range subsumed therein, including but not limited to, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL. In some aspects, the IN pharmaceutical formulation comprises naloxone HCl dihydrate present in an amount of about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, or about 4 mg/mL to about 5 mg/mL. In a preferred embodiment, the IN pharmaceutical formulation comprises naloxone HCl dihydrate present in an amount of about 30 mg/mL to about 50 mg/mL.

In other embodiments, the IN pharmaceutical formulation comprises naloxone HCl anhydrous present in an amount of about 5 mg/mL to about 50 mg/mL or any amount range subsumed therein, including but not limited to, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL.

In some embodiments, the IN pharmaceutical formulation comprises naloxone HCl anhydrous present in an amount of about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, or about 4 mg/mL to about 5 mg/mL. In a preferred embodiment, the IN pharmaceutical formulation comprises naloxone HCl anhydrous present in an amount of about 30 mg/mL to about 50 mg/mL.

In some embodiments, the IN pharmaceutical formulation comprises an opioid antagonist, or a hydrate thereof, present in an amount of about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 50 mg/mL or any amount range subsumed therein, including but not limited to, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL.

In a preferred embodiment, the IN pharmaceutical formulation comprises an opioid antagonist, or a hydrate thereof, present in an amount of about 30 mg/mL to about 50 mg/mL.

In some embodiments, the IN pharmaceutical formulation comprises an anhydrous opioid antagonist present in an amount of about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 50 mg/mL or any amount range subsumed therein, including but not limited to, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL.

In a preferred embodiment, the IN pharmaceutical formulation comprises an anhydrous opioid antagonist present in an amount of about 30 mg/mL to about 50 mg/mL.

In some embodiments, the IN pharmaceutical formulation comprises a pharmaceutically acceptable salt of an opioid antagonist present in an amount of about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 50 mg/mL or any amount range subsumed therein, including but not limited to, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL.

In a preferred embodiment, the IN pharmaceutical formulation comprises a pharmaceutically acceptable salt of an opioid antagonist present in an amount of about 30 mg/mL to about 50 mg/mL.

In some embodiments, the IN pharmaceutical formulation comprises naloxone, or a hydrate thereof, present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg.

In a preferred embodiment, the IN pharmaceutical formulation comprises naloxone, or a hydrate thereof, present in a dose amount of about 0.5 mg to about 5 mg.

In other embodiments, the IN pharmaceutical formulation comprises naloxone HCl dihydrate present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg. In a preferred embodiment, the IN pharmaceutical formulation comprises naloxone HCl dihydrate present in a dose amount of about 0.5 mg to about 5 mg.

In still other embodiments, the IN pharmaceutical formulation comprises naloxone HCl anhydrous present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg.

In a preferred embodiment, the IN pharmaceutical formulation comprises naloxone HCl anhydrous present in a dose amount of about 0.5 mg to about 5 mg.

In some embodiments, the IN pharmaceutical formulation comprises an opioid antagonist, or a hydrate thereof, present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg.

In a preferred embodiment, the IN pharmaceutical formulation comprises an opioid antagonist, or a hydrate thereof, present in a dose amount of about 0.5 mg to about 5 mg.

In some embodiments, the IN pharmaceutical formulation comprises an anhydrous opioid antagonist present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg.

In a preferred embodiment, the IN pharmaceutical formulation comprises an anhydrous opioid antagonist present in a dose amount of about 0.5 mg to about 5 mg.

In some embodiments, the IN pharmaceutical formulation comprises a pharmaceutically acceptable salt of an opioid antagonist present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg.

In a preferred embodiment, the IN pharmaceutical formulation comprises a pharmaceutically acceptable salt of an opioid antagonist present in a dose amount of about 0.5 mg to about 5 mg.

In some embodiments, the pH of the IN naloxone, or a hydrate thereof, pharmaceutical formulation is about 3 to about 5, or any pH range subsumed therein, including but not limited to, about 3 to about 4.8, about 3 to about 4.5, about 3 to about 4.3, about 3 to about 4, about 3 to about 3.8, about 3 to about 3.5, about 3.3 to about 5, about 3.3 to about 4.8, about 3.3 to about 4.5, about 3.3 to about 4.3, about 3.3 to about 4, about 3.3 to about 3.8, about 3.3 to about 3.5, about 3.5 to about 5, about 3.5 to about 4.8, about 3.5 to about 4.5, about 3.5 to about 4.3, about 3.5 to about 4, about 3.5 to about 3.8, about 3.8 to about 5, about 3.8 to about 4.8, about 3.8 to about 4.5, about 3.8 to about 4.3, or about 3.8 to about 4. In other embodiments, the pH of the IN naloxone, or hydrate thereof, pharmaceutical formulation is about 3, about 3.1, about 3.2, 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5.

In a preferred embodiment, the pH of the IN naloxone, or a hydrate thereof, pharmaceutical formulation is about 4 to about 5.

In some embodiments, the pH of the IN naloxone HCl dihydrate pharmaceutical formulation is about 3 to about 5, or any pH range subsumed therein, including but not limited to, about 3 to about 4.8, about 3 to about 4.5, about 3 to about 4.3, about 3 to about 4, about 3 to about 3.8, about 3 to about 3.5, about 3.3 to about 5, about 3.3 to about 4.8, about 3.3 to about 4.5, about 3.3 to about 4.3, about 3.3 to about 4, about 3.3 to about 3.8, about 3.3 to about 3.5, about 3.5 to about 5, about 3.5 to about 4.8, about 3.5 to about 4.5, about 3.5 to about 4.3, about 3.5 to about 4, about 3.5 to about 3.8, about 3.8 to about 5, about 3.8 to about 4.8, about 3.8 to about 4.5, about 3.8 to about 4.3, or about 3.8 to about 4. In other embodiments, the pH of the IN naloxone HCl dihydrate pharmaceutical formulation is about 3, about 3.1, about 3.2, 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5.

In a preferred embodiment, the pH of the IN naloxone HCl dihydrate pharmaceutical formulation is about 4 to about 5.

In some embodiments, the pH of the IN naloxone HCl anhydrous pharmaceutical formulation is about 3 to about 5, or any pH range subsumed therein, including but not limited to, about 3 to about 4.8, about 3 to about 4.5, about 3 to about 4.3, about 3 to about 4, about 3 to about 3.8, about 3 to about 3.5, about 3.3 to about 5, about 3.3 to about 4.8, about 3.3 to about 4.5, about 3.3 to about 4.3, about 3.3 to about 4, about 3.3 to about 3.8, about 3.3 to about 3.5, about 3.5 to about 5, about 3.5 to about 4.8, about 3.5 to about 4.5, about 3.5 to about 4.3, about 3.5 to about 4, about 3.5 to about 3.8, about 3.8 to about 5, about 3.8 to about 4.8, about 3.8 to about 4.5, about 3.8 to about 4.3, or about 3.8 to about 4. In other embodiments, the pH of the IN naloxone HCl anhydrous pharmaceutical formulation is about 3, about 3.1, about 3.2, 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5.

In a preferred embodiment, the pH of the IN naloxone HCl anhydrous pharmaceutical formulation is about 4 to about 5.

In some embodiments, the pH of the IN opioid antagonist, or hydrate thereof, pharmaceutical formulation is about 3 to about 5, or any pH range subsumed therein, including but not limited to, about 3 to about 4.8, about 3 to about 4.5, about 3 to about 4.3, about 3 to about 4, about 3 to about 3.8, about 3 to about 3.5, about 3.3 to about 5, about 3.3 to about 4.8, about 3.3 to about 4.5, about 3.3 to about 4.3, about 3.3 to about 4, about 3.3 to about 3.8, about 3.3 to about 3.5, about 3.5 to about 5, about 3.5 to about 4.8, about 3.5 to about 4.5, about 3.5 to about 4.3, about 3.5 to about 4, about 3.5 to about 3.8, about 3.8 to about 5, about 3.8 to about 4.8, about 3.8 to about 4.5, about 3.8 to about 4.3, or about 3.8 to about 4. In other embodiments, the pH of the IN opioid antagonist pharmaceutical formulation is about 3, about 3.1, about 3.2, 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5.

In a preferred embodiment, the pH of the IN opioid antagonist, or hydrate thereof, pharmaceutical formulation is about 4 to about 5.

In some embodiments, the pH of the IN anhydrous opioid antagonist pharmaceutical formulation is about 3 to about 5, or any pH range subsumed therein, including but not limited to, about 3 to about 4.8, about 3 to about 4.5, about 3 to about 4.3, about 3 to about 4, about 3 to about 3.8, about 3 to about 3.5, about 3.3 to about 5, about 3.3 to about 4.8, about 3.3 to about 4.5, about 3.3 to about 4.3, about 3.3 to about 4, about 3.3 to about 3.8, about 3.3 to about 3.5, about 3.5 to about 5, about 3.5 to about 4.8, about 3.5 to about 4.5, about 3.5 to about 4.3, about 3.5 to about 4, about 3.5 to about 3.8, about 3.8 to about 5, about 3.8 to about 4.8, about 3.8 to about 4.5, about 3.8 to about 4.3, or about 3.8 to about 4. In other embodiments, the pH of the IN anhydrous opioid antagonist pharmaceutical formulation is about 3, about 3.1, about 3.2, 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5.

In a preferred embodiment, the pH of the IN anhydrous opioid antagonist pharmaceutical formulation is about 4 to about 5.

In some embodiments, the pH of the IN pharmaceutically acceptable salt of an opioid antagonist pharmaceutical formulation is about 3 to about 5, or any pH range subsumed therein, including but not limited to, about 3 to about 4.8, about 3 to about 4.5, about 3 to about 4.3, about 3 to about 4, about 3 to about 3.8, about 3 to about 3.5, about 3.3 to about 5, about 3.3 to about 4.8, about 3.3 to about 4.5, about 3.3 to about 4.3, about 3.3 to about 4, about 3.3 to about 3.8, about 3.3 to about 3.5, about 3.5 to about 5, about 3.5 to about 4.8, about 3.5 to about 4.5, about 3.5 to about 4.3, about 3.5 to about 4, about 3.5 to about 3.8, about 3.8 to about 5, about 3.8 to about 4.8, about 3.8 to about 4.5, about 3.8 to about 4.3, or about 3.8 to about 4. In other embodiments, the pH of the IN anhydrous opioid antagonist pharmaceutical formulation is about 3, about 3.1, about 3.2, 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, or about 5.

In a preferred embodiment, the pH of the IN pharmaceutically acceptable salt of an opioid antagonist pharmaceutical formulation is about 4 to about 5.

In some embodiments, the IN naloxone, or hydrate thereof, pharmaceutical formulation comprises a second active agent. In some embodiments, the second active agent is buprenorphine, or a pharmaceutically acceptable salt thereof. In some embodiments, the second active agent is methadone.

In some embodiments, the IN naloxone, or hydrate thereof, pharmaceutical formulation comprises naloxone, or a hydrate thereof, and a second active agent, such as buprenorphine, wherein the ratio between the naloxone, or a hydrate thereof, and the second active agent is about 10:1, about 9:1, about 8:1, about 7:1, about 6:1, about 5:1, about 4:1, about 3:1, about 3:2, about 2:1, about 1:1, about 1:2, about 1:3, about 2:3, about 1:4, about 1:5, about 1:6, about 1:7, about 1:8, about 1:9, or about 1:10.

In some embodiments, the IN naloxone, or hydrate thereof, pharmaceutical formulation comprises a second active agent, such as buprenorphine, present in an amount of about 5 mg/mL to about 50 mg/mL or any amount range subsumed therein, including but not limited to, about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL.

In some embodiments, the IN naloxone, or hydrate thereof, pharmaceutical formulation comprises a second active agent, such as buprenorphine, present in an amount of about 50 mg/mL to about 60 mg/mL, about 50 mg/mL to about 70 mg/mL, about 50 mg/mL to about 80 mg/mL, about 50 mg/mL to about 90 mg/mL, about 50 mg/mL to about 100 mg/mL, about 50 mg/mL to about 120 mg/mL, about 50 mg/mL to about 140 mg/mL, about 50 mg/mL to about 160 mg/mL, about 50 mg/mL to about 180 mg/mL, about 50 mg/mL to about 200 mg/mL, about 60 mg/mL to about 70 mg/mL, about 60 mg/mL to about 80 mg/mL, about 60 mg/mL to about 90 mg/mL, about 60 mg/mL to about 100 mg/mL, about 60 mg/mL to about 120 mg/mL, about 60 mg/mL to about 140 mg/mL, about 60 mg/mL to about 160 mg/mL, about 60 mg/mL to about 180 mg/mL, about 60 mg/mL to about 200 mg/mL, about 70 mg/mL to about 7 mg/mL, about 70 mg/mL to about 90 mg/mL, about 70 mg/mL to about 100 mg/mL, about 70 mg/mL to about 120 mg/mL, about 70 mg/mL to about 140 mg/mL, about 70 mg/mL to about 160 mg/mL, about 70 mg/mL to about 180 mg/mL, about 70 mg/mL to about 200 mg/mL, about 80 mg/mL to about 90 mg/mL, about 80 mg/mL to about 100 mg/mL, about 80 mg/mL to about 120 mg/mL, about 80 mg/mL to about 140 mg/mL, about 80 mg/mL to about 160 mg/mL, about 80 mg/mL to about 180 mg/mL, about 80 mg/mL to about 200 mg/mL, about 90 mg/mL to about 100 mg/mL, about 90 mg/mL to about 120 mg/mL, about 90 mg/mL to about 140 mg/mL, about 90 mg/mL to about 160 mg/mL, about 90 mg/mL to about 180 mg/mL, about 90 mg/mL to about 200 mg/mL, about 100 mg/mL to about 120 mg/mL, about 100 mg/mL to about 140 mg/mL, about 100 mg/mL to about 160 mg/mL, about 100 mg/mL to about 180 mg/mL, about 100 mg/mL to about 200 mg/mL, about 120 mg/mL to about 140 mg/mL, about 120 mg/mL to about 160 mg/mL, about 120 mg/mL to about 180 mg/mL, about 120 mg/mL to about 200 mg/mL, about 140 mg/mL to about 160 mg/mL, about 140 mg/mL to about 180 mg/mL, about 140 mg/mL to about 200 mg/mL, about 160 mg/mL to about 180 mg/mL, about 160 mg/mL to about 200 mg/mL, or about 180 mg/mL to about 200 mg/mL.

In some embodiments, the IN naloxone, or hydrate thereof, pharmaceutical formulation comprises a second active agent, such as buprenorphine, present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg. In some embodiments, the IN naloxone, or hydrate thereof, pharmaceutical formulation comprises a second active agent, such as buprenorphine, present in a dose amount of about 15 mg to about 20 mg, about 15 mg to about 25 mg, about 15 mg to about 30 mg, about 15 mg to about 35 mg, about 15 mg to about 40 mg, about 20 mg to about 25 mg, about 20 mg to about 30 mg, about 20 mg to about 35 mg, about 20 mg to about 40 mg, about 25 mg to about 30 mg, about 25 mg to about 35 mg, about 25 mg to about 40 mg, about 30 mg to about 35 mg, about 30 mg to about 40 mg, or about 35 mg to about 40 mg.

In some embodiments, the IN naloxone HCl dihydrate pharmaceutical formulation comprises a second active agent. In some embodiments, the second active agent is buprenorphine, or a pharmaceutically acceptable salt thereof. In some embodiments, the second active agent is methadone.

In some embodiments, the IN naloxone HCl dihydrate pharmaceutical formulation comprises naloxone HCl dihydrate and a second active agent, such as buprenorphine, wherein the ratio between the naloxone HCl dihydrate and the second active agent is about 10:1, about 9:1, about 8:1, about 7:1, about 6:1, about 5:1, about 4:1, about 3:1, about 3:2, about 2:1, about 1:1, about 1:2, about 1:3, about 2:3, about 1:4, about 1:5, about 1:6, about 1:7, about 1:8, about 1:9, or about 1:10.

In some embodiments, the IN naloxone HCl dihydrate pharmaceutical formulation comprises a second active agent, such as buprenorphine, present in an amount of about 5 mg/mL to about 50 mg/mL or any amount range subsumed therein, including but not limited to, about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL. In some embodiments, the IN naloxone HCl dihydate pharmaceutical formulation comprises a second active agent, such as buprenorphine, present in an amount of about 50 mg/mL to about 60 mg/mL, about 50 mg/mL to about 70 mg/mL, about 50 mg/mL to about 80 mg/mL, about 50 mg/mL to about 90 mg/mL, about 50 mg/mL to about 100 mg/mL, about 50 mg/mL to about 120 mg/mL, about 50 mg/mL to about 140 mg/mL, about 50 mg/mL to about 160 mg/mL, about 50 mg/mL to about 180 mg/mL, about 50 mg/mL to about 200 mg/mL, about 60 mg/mL to about 70 mg/mL, about 60 mg/mL to about 80 mg/mL, about 60 mg/mL to about 90 mg/mL, about 60 mg/mL to about 100 mg/mL, about 60 mg/mL to about 120 mg/mL, about 60 mg/mL to about 140 mg/mL, about 60 mg/mL to about 160 mg/mL, about 60 mg/mL to about 180 mg/mL, about 60 mg/mL to about 200 mg/mL, about 70 mg/mL to about 7 mg/mL, about 70 mg/mL to about 90 mg/mL, about 70 mg/mL to about 100 mg/mL, about 70 mg/mL to about 120 mg/mL, about 70 mg/mL to about 140 mg/mL, about 70 mg/mL to about 160 mg/mL, about 70 mg/mL to about 180 mg/mL, about 70 mg/mL to about 200 mg/mL, about 80 mg/mL to about 90 mg/mL, about 80 mg/mL to about 100 mg/mL, about 80 mg/mL to about 120 mg/mL, about 80 mg/mL to about 140 mg/mL, about 80 mg/mL to about 160 mg/mL, about 80 mg/mL to about 180 mg/mL, about 80 mg/mL to about 200 mg/mL, about 90 mg/mL to about 100 mg/mL, about 90 mg/mL to about 120 mg/mL, about 90 mg/mL to about 140 mg/mL, about 90 mg/mL to about 160 mg/mL, about 90 mg/mL to about 180 mg/mL, about 90 mg/mL to about 200 mg/mL, about 100 mg/mL to about 120 mg/mL, about 100 mg/mL to about 140 mg/mL, about 100 mg/mL to about 160 mg/mL, about 100 mg/mL to about 180 mg/mL, about 100 mg/mL to about 200 mg/mL, about 120 mg/mL to about 140 mg/mL, about 120 mg/mL to about 160 mg/mL, about 120 mg/mL to about 180 mg/mL, about 120 mg/mL to about 200 mg/mL, about 140 mg/mL to about 160 mg/mL, about 140 mg/mL to about 180 mg/mL, about 140 mg/mL to about 200 mg/mL, about 160 mg/mL to about 180 mg/mL, about 160 mg/mL to about 200 mg/mL, or about 180 mg/mL to about 200 mg/mL.

In some embodiments, the IN naloxone HCl dihydrate pharmaceutical formulation comprises a second active agent, such as buprenorphine, present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg. In some embodiments, the IN naloxone HCl dihydrate pharmaceutical formulation comprises a second active agent, such as buprenorphine, present in a dose amount of about 15 mg to about 20 mg, about 15 mg to about 25 mg, about 15 mg to about 30 mg, about 15 mg to about 35 mg, about 15 mg to about 40 mg, about 20 mg to about 25 mg, about 20 mg to about 30 mg, about 20 mg to about 35 mg, about 20 mg to about 40 mg, about 25 mg to about 30 mg, about 25 mg to about 35 mg, about 25 mg to about 40 mg, about 30 mg to about 35 mg, about 30 mg to about 40 mg, or about 35 mg to about 40 mg.

In some embodiments, the IN naloxone HCl anhydrous pharmaceutical formulation comprises a second active agent. In some embodiments, the second active agent is buprenorphine, or a pharmaceutically acceptable salt thereof. In some embodiments, the second active agent is methadone.

In some embodiments, the IN naloxone HCl anhydrous pharmaceutical formulation comprises naloxone HCl anhydrous and a second active agent, such as buprenorphine, wherein the ratio between the naloxone HCl anhydrous and the second active agent is about 10:1, about 9:1, about 8:1, about 7:1, about 6:1, about 5:1, about 4:1, about 3:1, about 3:2, about 2:1, about 1:1, about 1:2, about 1:3, about 2:3, about 1:4, about 1:5, about 1:6, about 1:7, about 1:8, about 1:9, or about 1:10.

In some embodiments, the IN naloxone HCl anhydrous pharmaceutical formulation comprises a second active agent, such as buprenorphine, present in an amount of about 5 mg/mL to about 50 mg/mL or any amount range subsumed therein, including but not limited to, about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL. In some embodiments, the IN naloxone HCl anhydrous pharmaceutical formulation comprises a second active agent, such as buprenorphine, present in an amount of about 50 mg/mL to about 60 mg/mL, about 50 mg/mL to about 70 mg/mL, about 50 mg/mL to about 80 mg/mL, about 50 mg/mL to about 90 mg/mL, about 50 mg/mL to about 100 mg/mL, about 50 mg/mL to about 120 mg/mL, about 50 mg/mL to about 140 mg/mL, about 50 mg/mL to about 160 mg/mL, about 50 mg/mL to about 180 mg/mL, about 50 mg/mL to about 200 mg/mL, about 60 mg/mL to about 70 mg/mL, about 60 mg/mL to about 80 mg/mL, about 60 mg/mL to about 90 mg/mL, about 60 mg/mL to about 100 mg/mL, about 60 mg/mL to about 120 mg/mL, about 60 mg/mL to about 140 mg/mL, about 60 mg/mL to about 160 mg/mL, about 60 mg/mL to about 180 mg/mL, about 60 mg/mL to about 200 mg/mL, about 70 mg/mL to about 7 mg/mL, about 70 mg/mL to about 90 mg/mL, about 70 mg/mL to about 100 mg/mL, about 70 mg/mL to about 120 mg/mL, about 70 mg/mL to about 140 mg/mL, about 70 mg/mL to about 160 mg/mL, about 70 mg/mL to about 180 mg/mL, about 70 mg/mL to about 200 mg/mL, about 80 mg/mL to about 90 mg/mL, about 80 mg/mL to about 100 mg/mL, about 80 mg/mL to about 120 mg/mL, about 80 mg/mL to about 140 mg/mL, about 80 mg/mL to about 160 mg/mL, about 80 mg/mL to about 180 mg/mL, about 80 mg/mL to about 200 mg/mL, about 90 mg/mL to about 100 mg/mL, about 90 mg/mL to about 120 mg/mL, about 90 mg/mL to about 140 mg/mL, about 90 mg/mL to about 160 mg/mL, about 90 mg/mL to about 180 mg/mL, about 90 mg/mL to about 200 mg/mL, about 100 mg/mL to about 120 mg/mL, about 100 mg/mL to about 140 mg/mL, about 100 mg/mL to about 160 mg/mL, about 100 mg/mL to about 180 mg/mL, about 100 mg/mL to about 200 mg/mL, about 120 mg/mL to about 140 mg/mL, about 120 mg/mL to about 160 mg/mL, about 120 mg/mL to about 180 mg/mL, about 120 mg/mL to about 200 mg/mL, about 140 mg/mL to about 160 mg/mL, about 140 mg/mL to about 180 mg/mL, about 140 mg/mL to about 200 mg/mL, about 160 mg/mL to about 180 mg/mL, about 160 mg/mL to about 200 mg/mL, or about 180 mg/mL to about 200 mg/mL.

In some embodiments, the IN naloxone HCl anhydrous pharmaceutical formulation comprises a second active agent, such as buprenorphine, present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg. In some embodiments, the IN naloxone HCl anhydrous pharmaceutical formulation comprises a second active agent, such as buprenorphine, present in a dose amount of about 15 mg to about 20 mg, about 15 mg to about 25 mg, about 15 mg to about 30 mg, about 15 mg to about 35 mg, about 15 mg to about 40 mg, about 20 mg to about 25 mg, about 20 mg to about 30 mg, about 20 mg to about 35 mg, about 20 mg to about 40 mg, about 25 mg to about 30 mg, about 25 mg to about 35 mg, about 25 mg to about 40 mg, about 30 mg to about 35 mg, about 30 mg to about 40 mg, or about 35 mg to about 40 mg.

In some embodiments, the IN opioid antagonist, or hydrate thereof, pharmaceutical formulation comprises a second active agent. In some embodiments, the second active agent is buprenorphine, or a pharmaceutically acceptable salt thereof. In some embodiments, the second active agent is methadone.

In some embodiments, the IN opioid antagonist, or hydrate thereof, pharmaceutical formulation comprises an opioid antagonist, or hydrate thereof, and a second active agent, such as buprenorphine, wherein the ratio between the opioid antagonist, or hydrate thereof, and the second active agent is about 10:1, about 9:1, about 8:1, about 7:1, about 6:1, about 5:1, about 4:1, about 3:1, about 3:2, about 2:1, about 1:1, about 1:2, about 1:3, about 2:3, about 1:4, about 1:5, about 1:6, about 1:7, about 1:8, about 1:9, or about 1:10.

In some embodiments, the IN opioid antagonist, or hydrate thereof, pharmaceutical formulation comprises a second active agent, such as buprenorphine, present in an amount of about 5 mg/mL to about 50 mg/mL or any amount range subsumed therein, including but not limited to, about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL. In some embodiments, the IN opioid antagonist, or hydrate thereof, pharmaceutical formulation comprises a second active agent, such as buprenorphine, present in an amount of about 50 mg/mL to about 60 mg/mL, about 50 mg/mL to about 70 mg/mL, about 50 mg/mL to about 80 mg/mL, about 50 mg/mL to about 90 mg/mL, about 50 mg/mL to about 100 mg/mL, about 50 mg/mL to about 120 mg/mL, about 50 mg/mL to about 140 mg/mL, about 50 mg/mL to about 160 mg/mL, about 50 mg/mL to about 180 mg/mL, about 50 mg/mL to about 200 mg/mL, about 60 mg/mL to about 70 mg/mL, about 60 mg/mL to about 80 mg/mL, about 60 mg/mL to about 90 mg/mL, about 60 mg/mL to about 100 mg/mL, about 60 mg/mL to about 120 mg/mL, about 60 mg/mL to about 140 mg/mL, about 60 mg/mL to about 160 mg/mL, about 60 mg/mL to about 180 mg/mL, about 60 mg/mL to about 200 mg/mL, about 70 mg/mL to about 7 mg/mL, about 70 mg/mL to about 90 mg/mL, about 70 mg/mL to about 100 mg/mL, about 70 mg/mL to about 120 mg/mL, about 70 mg/mL to about 140 mg/mL, about 70 mg/mL to about 160 mg/mL, about 70 mg/mL to about 180 mg/mL, about 70 mg/mL to about 200 mg/mL, about 80 mg/mL to about 90 mg/mL, about 80 mg/mL to about 100 mg/mL, about 80 mg/mL to about 120 mg/mL, about 80 mg/mL to about 140 mg/mL, about 80 mg/mL to about 160 mg/mL, about 80 mg/mL to about 180 mg/mL, about 80 mg/mL to about 200 mg/mL, about 90 mg/mL to about 100 mg/mL, about 90 mg/mL to about 120 mg/mL, about 90 mg/mL to about 140 mg/mL, about 90 mg/mL to about 160 mg/mL, about 90 mg/mL to about 180 mg/mL, about 90 mg/mL to about 200 mg/mL, about 100 mg/mL to about 120 mg/mL, about 100 mg/mL to about 140 mg/mL, about 100 mg/mL to about 160 mg/mL, about 100 mg/mL to about 180 mg/mL, about 100 mg/mL to about 200 mg/mL, about 120 mg/mL to about 140 mg/mL, about 120 mg/mL to about 160 mg/mL, about 120 mg/mL to about 180 mg/mL, about 120 mg/mL to about 200 mg/mL, about 140 mg/mL to about 160 mg/mL, about 140 mg/mL to about 180 mg/mL, about 140 mg/mL to about 200 mg/mL, about 160 mg/mL to about 180 mg/mL, about 160 mg/mL to about 200 mg/mL, or about 180 mg/mL to about 200 mg/mL.

In some embodiments, the IN opioid antagonist, or hydrate thereof, pharmaceutical formulation comprises a second active agent, such as buprenorphine, present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg. In some embodiments, the IN opioid antagonist, or hydrate thereof, pharmaceutical formulation comprises a second active agent, such as buprenorphine, present in a dose amount of about 15 mg to about 20 mg, about 15 mg to about 25 mg, about 15 mg to about 30 mg, about 15 mg to about 35 mg, about 15 mg to about 40 mg, about 20 mg to about 25 mg, about 20 mg to about 30 mg, about 20 mg to about 35 mg, about 20 mg to about 40 mg, about 25 mg to about 30 mg, about 25 mg to about 35 mg, about 25 mg to about 40 mg, about 30 mg to about 35 mg, about 30 mg to about 40 mg, or about 35 mg to about 40 mg.

In some embodiments, the IN anhydrous opioid antagonist pharmaceutical formulation comprises a second active agent. In some embodiments, the second active agent is buprenorphine, or a pharmaceutically acceptable salt thereof. In some embodiments, the second active agent is methadone.

In some embodiments, the IN anhydrous opioid antagonist pharmaceutical formulation comprises an anhydrous opioid antagonist and a second active agent, such as buprenorphine, wherein the ratio between the anhydrous opioid antagonist and the second active agent is about 10:1, about 9:1, about 8:1, about 7:1, about 6:1, about 5:1, about 4:1, about 3:1, about 3:2, about 2:1, about 1:1, about 1:2, about 1:3, about 2:3, about 1:4, about 1:5, about 1:6, about 1:7, about 1:8, about 1:9, or about 1:10.

In some embodiments, the IN anhydrous opioid antagonist pharmaceutical formulation comprises a second active agent, such as buprenorphine, present in an amount of about 5 mg/mL to about 50 mg/mL or any amount range subsumed therein, including but not limited to, about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL. In some embodiments, the IN anhydrous opioid antagonist pharmaceutical formulation comprises a second active agent, such as buprenorphine, present in an amount of about 50 mg/mL to about 60 mg/mL, about 50 mg/mL to about 70 mg/mL, about 50 mg/mL to about 80 mg/mL, about 50 mg/mL to about 90 mg/mL, about 50 mg/mL to about 100 mg/mL, about 50 mg/mL to about 120 mg/mL, about 50 mg/mL to about 140 mg/mL, about 50 mg/mL to about 160 mg/mL, about 50 mg/mL to about 180 mg/mL, about 50 mg/mL to about 200 mg/mL, about 60 mg/mL to about 70 mg/mL, about 60 mg/mL to about 80 mg/mL, about 60 mg/mL to about 90 mg/mL, about 60 mg/mL to about 100 mg/mL, about 60 mg/mL to about 120 mg/mL, about 60 mg/mL to about 140 mg/mL, about 60 mg/mL to about 160 mg/mL, about 60 mg/mL to about 180 mg/mL, about 60 mg/mL to about 200 mg/mL, about 70 mg/mL to about 7 mg/mL, about 70 mg/mL to about 90 mg/mL, about 70 mg/mL to about 100 mg/mL, about 70 mg/mL to about 120 mg/mL, about 70 mg/mL to about 140 mg/mL, about 70 mg/mL to about 160 mg/mL, about 70 mg/mL to about 180 mg/mL, about 70 mg/mL to about 200 mg/mL, about 80 mg/mL to about 90 mg/mL, about 80 mg/mL to about 100 mg/mL, about 80 mg/mL to about 120 mg/mL, about 80 mg/mL to about 140 mg/mL, about 80 mg/mL to about 160 mg/mL, about 80 mg/mL to about 180 mg/mL, about 80 mg/mL to about 200 mg/mL, about 90 mg/mL to about 100 mg/mL, about 90 mg/mL to about 120 mg/mL, about 90 mg/mL to about 140 mg/mL, about 90 mg/mL to about 160 mg/mL, about 90 mg/mL to about 180 mg/mL, about 90 mg/mL to about 200 mg/mL, about 100 mg/mL to about 120 mg/mL, about 100 mg/mL to about 140 mg/mL, about 100 mg/mL to about 160 mg/mL, about 100 mg/mL to about 180 mg/mL, about 100 mg/mL to about 200 mg/mL, about 120 mg/mL to about 140 mg/mL, about 120 mg/mL to about 160 mg/mL, about 120 mg/mL to about 180 mg/mL, about 120 mg/mL to about 200 mg/mL, about 140 mg/mL to about 160 mg/mL, about 140 mg/mL to about 180 mg/mL, about 140 mg/mL to about 200 mg/mL, about 160 mg/mL to about 180 mg/mL, about 160 mg/mL to about 200 mg/mL, or about 180 mg/mL to about 200 mg/mL.

In some embodiments, the IN anhydrous opioid antagonist pharmaceutical formulation comprises a second active agent, such as buprenorphine, present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg. In some embodiments, the IN anhydrous opioid antagonist pharmaceutical formulation comprises a second active agent, such as buprenorphine, present in a dose amount of about 15 mg to about 20 mg, about 15 mg to about 25 mg, about 15 mg to about 30 mg, about 15 mg to about 35 mg, about 15 mg to about 40 mg, about 20 mg to about 25 mg, about 20 mg to about 30 mg, about 20 mg to about 35 mg, about 20 mg to about 40 mg, about 25 mg to about 30 mg, about 25 mg to about 35 mg, about 25 mg to about 40 mg, about 30 mg to about 35 mg, about 30 mg to about 40 mg, or about 35 mg to about 40 mg.

In some embodiments, the IN pharmaceutically acceptable salt of an opioid antagonist pharmaceutical formulation comprises a second active agent. In some embodiments, the second active agent is buprenorphine, or a pharmaceutically acceptable salt thereof. In some embodiments, the second active agent is methadone.

In some embodiments, the IN pharmaceutically acceptable salt of an opioid antagonist pharmaceutical formulation comprises a pharmaceutically acceptable salt of an opioid antagonist and a second active agent, such as buprenorphine, wherein the ratio between the pharmaceutically acceptable salt of an opioid antagonist and the second active agent is about 10:1, about 9:1, about 8:1, about 7:1, about 6:1, about 5:1, about 4:1, about 3:1, about 3:2, about 2:1, about 1:1, about 1:2, about 1:3, about 2:3, about 1:4, about 1:5, about 1:6, about 1:7, about 1:8, about 1:9, or about 1:10.

In some embodiments, the IN pharmaceutically acceptable salt of an opioid antagonist pharmaceutical formulation comprises a second active agent, such as buprenorphine, present in an amount of about 5 mg/mL to about 50 mg/mL or any amount range subsumed therein, including but not limited to, about 1 mg/mL to about 2 mg/mL, about 1 mg/mL to about 3 mg/mL, about 1 mg/mL to about 4 mg/mL, about 1 mg/mL to about 5 mg/mL, about 2 mg/mL to about 3 mg/mL, about 2 mg/mL to about 4 mg/mL, about 2 mg/mL to about 5 mg/mL, about 3 mg/mL to about 4 mg/mL, about 3 mg/mL to about 5 mg/mL, about 4 mg/mL to about 5 mg/mL, about 5 mg/mL to about 45 mg/mL, about 5 mg/mL to about 40 mg/mL, about 5 mg/mL to about 35 mg/mL, about 5 mg/mL to about 30 mg/mL, about 5 mg/mL to about 25 mg/mL, about 5 mg/mL to about 20 mg/mL, about 5 mg/mL to about 15 mg/mL, about 10 mg/mL to about 50 mg/mL, about 10 mg/mL to about 45 mg/mL, about 10 mg/mL to about 40 mg/mL, about 10 mg/mL to about 35 mg/mL, about 10 mg/mL to about 30 mg/mL, about 10 mg/mL to about 25 mg/mL, about 10 mg/mL to about 20 mg/mL, about 10 mg/mL to about 15 mg/mL, about 15 mg/mL to about 50 mg/mL, about 15 mg/mL to about 45 mg/mL, about 15 mg/mL to about 40 mg/mL, about 15 mg/mL to about 35 mg/mL, about 15 mg/mL to about 30 mg/mL, about 15 mg/mL to about 25 mg/mL, about 15 mg/mL to about 20 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 25 mg/mL to about 50 mg/mL, about 25 mg/mL to about 45 mg/mL, about 25 mg/mL to about 40 mg/mL, about 25 mg/mL to about 35 mg/mL, about 25 mg/mL to about 30 mg/mL, about 20 mg/mL to about 50 mg/mL, about 20 mg/mL to about 45 mg/mL, about 20 mg/mL to about 40 mg/mL, about 20 mg/mL to about 35 mg/mL, about 20 mg/mL to about 30 mg/mL, about 20 mg/mL to about 25 mg/mL, about 30 mg/mL to about 50 mg/mL, about 35 mg/mL to about 45 mg/mL, about 40 mg/mL to about 50 mg/mL, about 40 mg/mL to about 45 mg/mL, or about 45 mg/mL to about 50 mg/mL. In some embodiments, the IN pharmaceutically acceptable salt of an opioid antagonist pharmaceutical formulation comprises a second active agent, such as buprenorphine, present in an amount of about 50 mg/mL to about 60 mg/mL, about 50 mg/mL to about 70 mg/mL, about 50 mg/mL to about 80 mg/mL, about 50 mg/mL to about 90 mg/mL, about 50 mg/mL to about 100 mg/mL, about 50 mg/mL to about 120 mg/mL, about 50 mg/mL to about 140 mg/mL, about 50 mg/mL to about 160 mg/mL, about 50 mg/mL to about 180 mg/mL, about 50 mg/mL to about 200 mg/mL, about 60 mg/mL to about 70 mg/mL, about 60 mg/mL to about 80 mg/mL, about 60 mg/mL to about 90 mg/mL, about 60 mg/mL to about 100 mg/mL, about 60 mg/mL to about 120 mg/mL, about 60 mg/mL to about 140 mg/mL, about 60 mg/mL to about 160 mg/mL, about 60 mg/mL to about 180 mg/mL, about 60 mg/mL to about 200 mg/mL, about 70 mg/mL to about 7 mg/mL, about 70 mg/mL to about 90 mg/mL, about 70 mg/mL to about 100 mg/mL, about 70 mg/mL to about 120 mg/mL, about 70 mg/mL to about 140 mg/mL, about 70 mg/mL to about 160 mg/mL, about 70 mg/mL to about 180 mg/mL, about 70 mg/mL to about 200 mg/mL, about 80 mg/mL to about 90 mg/mL, about 80 mg/mL to about 100 mg/mL, about 80 mg/mL to about 120 mg/mL, about 80 mg/mL to about 140 mg/mL, about 80 mg/mL to about 160 mg/mL, about 80 mg/mL to about 180 mg/mL, about 80 mg/mL to about 200 mg/mL, about 90 mg/mL to about 100 mg/mL, about 90 mg/mL to about 120 mg/mL, about 90 mg/mL to about 140 mg/mL, about 90 mg/mL to about 160 mg/mL, about 90 mg/mL to about 180 mg/mL, about 90 mg/mL to about 200 mg/mL, about 100 mg/mL to about 120 mg/mL, about 100 mg/mL to about 140 mg/mL, about 100 mg/mL to about 160 mg/mL, about 100 mg/mL to about 180 mg/mL, about 100 mg/mL to about 200 mg/mL, about 120 mg/mL to about 140 mg/mL, about 120 mg/mL to about 160 mg/mL, about 120 mg/mL to about 180 mg/mL, about 120 mg/mL to about 200 mg/mL, about 140 mg/mL to about 160 mg/mL, about 140 mg/mL to about 180 mg/mL, about 140 mg/mL to about 200 mg/mL, about 160 mg/mL to about 180 mg/mL, about 160 mg/mL to about 200 mg/mL, or about 180 mg/mL to about 200 mg/mL.

In some embodiments, the IN pharmaceutically acceptable salt of an opioid antagonist pharmaceutical formulation comprises a second active agent, such as buprenorphine, present in a dose amount of about 0.5 mg to about 20 mg, or any range subsumed therein, including but not limited to, about 0.5 mg to about 15 mg, about 0.5 mg to about 12 mg, about 0.5 mg to about 10 mg, about 0.5 mg to about 7.5 mg, about 0.5 mg to about 5 mg, about 0.5 mg to about 2.5 mg, about 1 mg to about 15 mg, about 1 mg to about 12.5 mg, about 1 mg to about 10 mg, about 1 mg to about 7.5 mg, about 1 mg to about 5 mg, about 1 mg to about 2.5 mg, about 2 mg to about 15 mg, about 2 mg to about 12.5 mg, about 2 mg to about 10 mg, about 2 mg to about 7.5 mg, about 2 mg to about 5 mg, about 3 mg to about 15 mg, about 3 mg to about 12.5 mg, about 3 mg to about 10 mg, about 3 mg to about 7.5 mg, about 3 mg to about 5 mg, about 4 mg to about 15 mg, about 4 mg to about 12.5 mg, about 4 mg to about 10 mg, about 4 mg to about 7.5 mg, about 5 mg to about 15 mg, about 5 mg to about 12.5 mg, about 5 mg to about 10 mg, about 5 mg to about 7.5 mg, about 6 mg to about 15 mg, about 6 mg to about 12.5 mg, about 6 mg to about 10 mg, about 6 mg to about 7.5 mg, about 7 mg to about 15 mg, about 7 mg to about 12.5 mg, about 7 mg to about 10 mg, about 8 mg to about 15 mg, about 8 mg to about 12.5 mg, about 8 mg to about 10 mg, about 9 mg to about 15 mg, about 9 mg to about 12.5 mg, about 10 mg to about 15 mg, about 10 mg to about 12.5 mg, about 11 mg to about 15 mg, about 12 to about 15 mg, or about 12.5 mg to about 15 mg. In some embodiments, the IN pharmaceutically acceptable salt of an opioid antagonist pharmaceutical formulation comprises a second active agent, such as buprenorphine, present in a dose amount of about 15 mg to about 20 mg, about 15 mg to about 25 mg, about 15 mg to about 30 mg, about 15 mg to about 35 mg, about 15 mg to about 40 mg, about 20 mg to about 25 mg, about 20 mg to about 30 mg, about 20 mg to about 35 mg, about 20 mg to about 40 mg, about 25 mg to about 30 mg, about 25 mg to about 35 mg, about 25 mg to about 40 mg, about 30 mg to about 35 mg, about 30 mg to about 40 mg, or about 35 mg to about 40 mg.

### IN Delivery Using a Nasal Spray

The disclosed IN naloxone and opioid antagonist pharmaceutical formulations can be administered by IN delivery using a nasal spray. Nasal sprays are commercially available to facilitate IN delivery of IN naloxone and opioid antagonist pharmaceutical formulations to one or more nostrils of a human patient. A nasal spray has a spray pump for discharging a dose volume of the IN naloxone and opioid antagonist pharmaceutical formulation in a single spray to a single nostril, or in two or more sprays to one or more nostrils. The dose volume of the IN formulation contains the dose amount of the naloxone, opioid antagonist, or a hydrate thereof, including, but not limited to, naloxone HCl dihydrate or naloxone HCl anhydrous, to be taken at one time. In the context of IN delivery, “to be taken at one time” covers the discharge of the dose volume in: (1) a single spray, or (2) two or more sprays in a very short amount of time, usually less than one minute.

In some embodiments, the nasal spray is a unit-dose nasal spray that administers a single dose volume of the IN pharmaceutical formulation in a single spray to a single nostril, or in two or more sprays to one or more nostrils, and such unit-dose nasal spray is disposed thereafter. In other embodiments, the nasal spray is a bi-dose nasal spray that can administer two dose volumes of the IN pharmaceutical formulation in two or more sprays to one or more nostrils, and such bi-dose nasal spray is disposed thereafter. In some embodiments, the unit-dose nasal spray or the bi-dose nasal spray is pre-primed to provide accurate dosing and ready to use capability. In still other embodiments, the nasal spray can administer three or more dose volumes of the IN pharmaceutical formulation.

Thus, in some embodiments, the dose amount of the naloxone, opioid antagonist, or a hydrate thereof, is discharged in a single spray. In other embodiments, the dose amount of the naloxone, opioid antagonist, or a hydrate thereof, is discharged in two or more sprays. In some embodiments, the dose amount of the naloxone, opioid antagonist, or a hydrate thereof, is discharged once. In some embodiments, the dose amount of the naloxone, opioid antagonist, or a hydrate thereof, is discharged 2, 3, 4, 5, or 6 times.

In some embodiments, the IN naloxone and opioid antagonist pharmaceutical formulation is an aqueous solution. In some embodiments, the aqueous solution comprises additional water-compatible solvents or additives. In some embodiments, the IN naloxone and opioid antagonist pharmaceutical formulation is in an aqueous solution having a dose volume of about 0.05 mL to about 0.5 mL, or any range subsumed therein, including but not limited to, about 0.1 mL to about 0.5 mL, about 0.15 mL to about 0.5 mL, about 0.2 mL to about 0.5 mL, about 0.25 mL to about 0.5 mL, about 0.05 mL to about 0.45 mL, about 0.05 mL to about 0.4 mL, about 0.05 mL to about 0.35 mL, about 0.05 mL to about 0.3 mL, about 0.05 mL to about 0.25 mL, about 0.05 mL to about 0.2 mL, about 0.05 mL to about 0.15 mL, about 0.1 mL to about 0.45 mL, about 0.1 mL to about 0.4 mL, about 0.1 mL to about 0.35 mL, about 0.1 mL to about 0.3 mL, about 0.1 mL to about 0.25 mL, about 0.15 mL to about 0.25 mL, about 0.1 mL to about 0.2 mL, about 0.1 mL to about 0.15 mL, about 0.15 mL to about 0.3 mL, about 0.15 mL to about 0.25 mL, or about 0.15 mL to about 0.2 mL. In other embodiments, the IN naloxone and opioid antagonist pharmaceutical formulation is in an aqueous solution having a dose volume of about 0.05 mL, about 0.1 mL, about 0.15 mL, about 0.2 mL, about 0.25 mL, about 0.3 mL, about 0.35 mL, about 0.4 mL, about 0.45 mL, or about 0.5 mL. The dose volume is the volume that contains the dose amount of the naloxone, opioid antagonist, or hydrate thereof.

### Bile Acids, and the Salts Thereof, are Absorption Enhancers of Naloxone by IN Delivery

The disclosed IN naloxone and opioid antagonist pharmaceutical formulations further comprise a bile acid, or the salt thereof, as an absorption enhancer. The term “absorption enhancer,” as used herein in the context of IN delivery, refers to an excipient in the IN pharmaceutical formulation whose function is to improve the absorption of the active agent into the bloodstream by enhancing permeation of the active agent through the nasal mucosa.

Bile acids and the salts thereof can enhance the IN absorption of drugs into the system circulation via the nasal mucosa. The nasal mucosa has two layers: (1) the outer epithelial layer, which is predominately lipophilic, and (2) the inner sublayer, known as the lamina propria, which comprises blood vessels for access to the bloodstream. Thus, the bile acids and the salts thereof can enhance the IN absorption of the naloxone by enabling the naloxone to access the blood vessels in the lamina propria of the nasal mucosa. After the active agent is absorbed into the bloodstream, the active agent can be distributed throughout the human body via the circulatory system.

Bile acids, and the salts thereof, are ionic amphiphilic compounds having a steroid skeleton. The physiological properties of bile acids and salts thereof include lipid transport by solubilization, transport of polar drugs through hydrophobic barriers, inhibition of enzyme activity, and opening tight junctions between epithelial cells. Within the human body, bile acids and salts thereof are amphipathic steroidal bio-surfactants that are derived from cholesterol in the liver. The synthesis of bile acids and salts thereof is the major route for elimination of cholesterol from the body.

While the present disclosure is not limited by any particular mechanism or theory, it is believed that based on the above-described properties, bile acids, and their salts, can enhance IN absorption by forming micelles, or reverse micelles, to enable transcellular passage of the naloxone or opioid antagonist through the predominantly lipophilic upper epithelial layer of the nasal mucosa and into the blood vessels located in the lamina propria sublayer. It is also believed that, in some embodiments, bile acids and their salts inhibit the tight junctions between the epithelial cells to enable paracellular passage of the naloxone or opioid antagonist through the predominantly lipophilic upper epithelial layer of the nasal mucosa and into the blood vessels located in the lamina propria sublayer. In this regard, bile acids and their salts can disrupt the hemidesmosomes or can bind to calcium in the tight junctions.

Additionally, bile acids and salts thereof can enhance IN absorption of naloxone or an opioid antagonist by inhibiting, degrading, or reducing enzymes, such as mucosal membrane peptidases, in the predominantly lipophilic upper epithelial layer of the nasal mucosa. Bile acids and salts thereof can enhance absorption of naloxone or an opioid antagonist by reducing the viscosity or elasticity of the predominantly lipophilic upper epithelial layer of the nasal mucosa. Therefore, bile acids and salts thereof can enhance IN absorptions of naloxone or an opioid antagonist through these aforementioned means or a combination thereof.

Bile acids and salts thereof can be formed when the conjugated bile acid complexes with sodium. Other suitable elements, such as potassium, may also be used to complex with the conjugate bile acid to form bile acids and salts thereof. Bile acids and salts thereof can be conjugated with an amino acid, such as glycine or taurine, to form conjugated bile acids and salts thereof. Bile acids and salts thereof are ionic amphiphilic compounds with a steroid skeleton.

The structure below is a non-limiting representative chemical structure of an embodiment of a bile acid or a salt thereof:

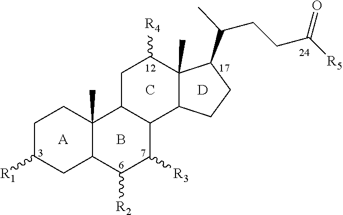

Wherein the numbering conforms to the steroid numbering system:

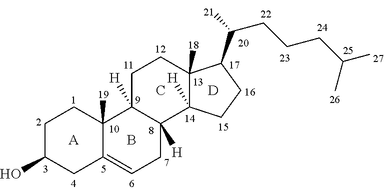

This example structure of a bile acid or a salt thereof consists of four rings, three six carbon rings (A, B and C), and one five carbon ring (D).

### Types of Bile Acids and Salts Thereof

Bile acids and their salts can be categorized into three main groups based on their conjugation with amino acids and their degree of hydroxylation. These three main groups are: (1) trihydroxy conjugates, (2) dihydroxy conjugates, and (3) unconjugated forms.

Exemplary embodiments of trihydroxy conjugates of bile acids and salts thereof include, but are not limited to, glycocholate (GC), taurocholate (TC), glycohyocholate (GHC), taurohyocholate (THC), tauro-α-muricholate (T-α-MC), tauro-β-muricholate (T-β-MC), or a combination thereof.

Exemplary embodiments of trihydroxy conjugates of bile acids and salts thereof include, but are not limited to, sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), or a combination thereof. Other suitable forms of salts are possible, such as substituting sodium with potassium (e.g. potassium glycocholate).

Exemplary embodiments of dihydroxy conjugates of bile acids and salts thereof include tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), or a combination thereof.

Exemplary embodiments of dihydroxy conjugates of bile acids and salts thereof include sodium tauroursodeoxycholate (STUDC), sodium taurohyodeoxycholate (STUDC), sodium glycohyodeoxycholate (SGUDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), or a combination thereof. Other suitable forms of salts are possible, such as substituting sodium with potassium (e.g. potassium tauroursodeoxycholate).

Exemplary embodiments of unconjugated forms of bile acids and salts thereof include cholate, deoxycholate (DC), chenodeoxycholate (CDC), or a combination thereof.

Exemplary embodiments of unconjugated forms of bile acids and salts thereof include sodium cholate (SC), sodium deoxycholate (SDC), sodium chenodeoxycholate (SCDC), or a combination thereof. Other suitable forms of salts are possible, such as substituting sodium with potassium (e.g. potassium cholate). The bile acids and salts thereof of the present disclosure are not limited to those described above, and may include any other suitable bile acids and salts thereof available in the art.

Bile acids and salts thereof can aggregate and form micelles in concentrations above the critical micelle concentration (CMC). By forming micelles, bile acids and salts thereof can facilitate transcellular passage and enhance absorption of naloxone or an opioid antagonist through the nasal mucosa. For example, CMC values of some bile acids and salts thereof are:
Sodium taurocholate (STC): CMC is ˜8 mM,
Sodium cholate (SC): CMC is ˜4 mM,
Sodium lithocholate (SLC): CMC is ˜1 mM,
Sodium glycocholate (SGC): CMC is ˜2-5 mM,
Sodium taurochenodeoxycholate (STCDC): CMC is ˜2.5-3 mM.

In some embodiments of the IN naloxone and opioid antagonist pharmaceutical formulations, the bile acid, or the salt thereof, is present at a concentration of about 1 mg/mL to about 15 mg/mL, or any concentration range subsumed therein, including but not limited to, about 1 mg/mL to about 12.5 mg/mL, about 1 mg/mL to about 10 mg/mL, about 4 mg/mL to about 12 mg/mL, about 5 mg/mL to about 11 mg/mL, about 6 mg/mL to about 13 mg/mL, about 7 mg/mL to about 12 mg/mL, about 7 mg/mL to about 9 mg/mL, about 7.5 mg/mL to about 9.5 mg/mL, about 7.5 mg/mL to about 8.5 mg/mL, about 7 mg/mL to about 9 mg/mL, or about 7 mg/mL to about 8 mg/mL. In still other embodiments of the naloxone and opioid antagonist pharmaceutical formulations, the bile acid, or the salt thereof, is present at a concentration of about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 10.5 mg/mL, about 11 mg/mL, about 11.5 mg/mL, about 12 mg/mL, about 12.5 mg/mL, about 13 mg/mL, about 13.5 mg/mL, about 14 mg/mL, about 14.5 mg/mL, or about 15 mg/mL.

In a preferred embodiment of the IN naloxone and opioid antagonist pharmaceutical formulations, the bile acid, or the salt thereof, is present at a concentration of 4 mg/mL to 12 mg/mL.

In some embodiments of the IN naloxone and opioid antagonist pharmaceutical formulations, the bile acid, or the salt thereof, is present in a dose amount of about 0.05 mg to about 1.5 mg, or any amount range subsumed therein, including but not limited to, about 0.6 mg to about 1.3 mg, about 0.5 mg to about 1.1 mg, about 0.7 mg to about 1.2 mg, about 0.7 mg to about 0.9 mg, about 0.75 mg to about 0.95 mg, about 0.75 mg to about 0.85 mg, about 0.70 mg to about 0.90 mg, or about 0.70 mg to about 0.80 mg. In still other embodiments of naloxone pharmaceutical formulations, the bile acid, or the salt thereof, is present in a dose amount of about 0.05 mg, about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, or about 1.5 mg.

In a preferred embodiment of the IN naloxone and opioid antagonist pharmaceutical formulations, the bile acid, or the salt thereof, is present at a concentration of about 0.05 mg to about 1 mg.

Thus, in some embodiments, the IN pharmaceutical formulation comprises naloxone, or a hydrate thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, buprenorphine, or a salt thereof, present in a dose amount of about 0.5 mg to about 40 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, buprenorphine, or a salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, buprenorphine, or a salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, buprenorphine, or a salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, buprenorphine, or a salt thereof, present in a dose amount of about 0.5 mg to about 40 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

Thus, in some embodiments, the IN pharmaceutical formulation comprises an opioid antagonist, or a hydrate or salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, a bile acid, or a salt thereof, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, buprenorphine, or a salt thereof, present in a dose amount of about 0.5 mg to about 40 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, a bile acid, or a salt thereof, present in a dose amount of about 0.1 mg to about 1.5 mg, wherein the IN pharmaceutical formulation has a pH of about 3 to about 5, and wherein the IN pharmaceutical formulation is for IN delivery.

In some embodiments, the ratio of the concentration of the active agent to the concentration of the bile acid, or salt thereof (active agent:bile acid or salt thereof) is about 50:1, about 45:1, about 40:1, about 35:1, about 30:1, about 25:1, about 20:1, about 15:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10.

In some embodiments, the degree of absorption enhancement of the bile acid, or salt thereof, is about 50%. In some embodiments, the degree of absorption enhancement is about 5%, about 10%, about 15%, about 20%, about 25%, about 30%, about 35%, about 40%, about 45%, about 55%, about 60%, about 65%, about 70%, about 75%, about 80%, about 85%, about 90%, about 95%, or about 100%.

### Exemplary Trihydroxy Conjugate Bile Acid or Salt Thereof: Sodium Taurocholate (STC)

As an exemplary embodiment of a trihydroxy conjugate bile acid, or salt thereof, sodium taurocholate (STC) is a trihydroxy conjugate bile salt that has the molecular formula C₂₆H₄₄NNaO₇S and a molecular weight (M.W.) of 537.7 g/mol. The chemical structure of STC is shown below:

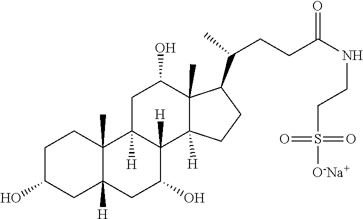

STC is an ionic amphiphilic compound with a steroid skeleton. It belongs to the family of endogenous bile acids and salts thereof, important for multiple physiological functions including lipid transport of nutrients and drugs across hydrophobic barriers through the process of solubilization. As shown by the chemical structure of STC, STC has a hydrophobic portion that includes the steroid portion, and a hydrophilic portion. STC has a critical micelle concentration (CMC) of about 4 mg/mL.

Thus, in some embodiments, the bile acid or salt thereof is an STC. In some embodiments, the STC is STC hydrate. In some embodiments of the IN naloxone and opioid antagonist pharmaceutical formulations, the STC is present at a concentration of about 1 mg/mL to about 15 mg/mL, or any concentration range subsumed therein, including but not limited to, about 1 mg/mL to about 12.5 mg/mL, about 1 mg/mL to about 10 mg/mL, about 4 mg/mL to about 12 mg/mL, about 5 mg/mL to about 11 mg/mL, about 6 mg/mL to about 13 mg/mL, about 7 mg/mL to about 12 mg/mL, about 7 mg/mL to about 9 mg/mL, about 7.5 mg/mL to about 9.5 mg/mL, about 7.5 mg/mL to about 8.5 mg/mL, about 7 mg/mL to about 9 mg/mL, or about 7 mg/mL to about 8 mg/mL. In still other embodiments of the IN naloxone pharmaceutical formulations, the bile acid, or the salt thereof, is present at a concentration of about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 10.5 mg/mL, about 11 mg/mL, about 11.5 mg/mL, about 12 mg/mL, about 12.5 mg/mL, about 13 mg/mL, about 13.5 mg/mL, about 14 mg/mL, about 14.5 mg/mL, or about 15 mg/mL.

In a preferred embodiment of the IN naloxone and opioid antagonist pharmaceutical formulations, the bile acid, or salt thereof, is STC present at a concentration of about 4 mg/mL to about 12 mg/mL.

In some embodiments of the IN naloxone and opioid antagonist pharmaceutical formulations, the STC is present in a dose amount of about 0.05 mg to about 1.5 mg, or any amount range subsumed therein, including but not limited to, about 0.6 mg to about 1.3 mg, about 0.5 mg to about 1.1 mg, about 0.7 mg to about 1.2 mg, about 0.7 mg to about 0.9 mg, about 0.75 mg to about 0.95 mg, about 0.75 mg to about 0.85 mg, about 0.70 mg to about 0.90 mg, or about 0.70 mg to about 0.80 mg. In still other embodiments of the IN naloxone and opioid antagonist pharmaceutical formulations, the bile acid, or the salt thereof, is present in a dose amount of about 0.05 mg, about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, or about 1.5 mg.

In a preferred embodiment of the IN naloxone and opioid antagonist pharmaceutical formulations, the bile acid, or salt thereof, is STC present in a dose amount of about 0.05 mg to about 1 mg.

Thus, in some embodiments, the IN naloxone pharmaceutical formulation comprises naloxone, or a hydrate thereof, present at a concentration of about 5 mg/mL to about 50 mg/mL, an absorption enhancer comprising STC, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN naloxone pharmaceutical formulation consists essentially of naloxone, or a hydrate thereof, present at a concentration of about 5 mg/mL to about 50 mg/mL, an absorption enhancer comprising STC, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN naloxone pharmaceutical formulation consists of naloxone, or a hydrate thereof, present at a concentration of about 5 mg/mL to about 50 mg/mL, an absorption enhancer comprising STC, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN naloxone pharmaceutical formulation comprises naloxone, or a hydrate thereof, present in a dose amount of about 0.5 mg to about 20 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN naloxone pharmaceutical formulation consists essentially of naloxone, or a hydrate thereof, present in a dose amount of about 0.5 mg to about 20 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN naloxone pharmaceutical formulation consists of naloxone, or a hydrate thereof, present in a dose amount of about 0.5 mg to about 20 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, buprenorphine, or a salt thereof, present in a dose amount of about 0.5 mg to about 40 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, buprenorphine, or a salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, buprenorphine, or a salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, buprenorphine, or a salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, buprenorphine, or a salt thereof, present in a dose amount of about 0.5 mg to about 40 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, STC present in a dose amount of about 0.05 mg to about 1.5 mg, wherein the IN pharmaceutical formulation has a pH of about 3 to about 5, and wherein the IN pharmaceutical formulation is for IN delivery.

Thus, in some embodiments, the IN pharmaceutical formulation comprises an opioid antagonist, or a hydrate or salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, STC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, buprenorphine, or a salt thereof, present in a dose amount of about 0.5 mg to about 40 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, STC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, STC present in a dose amount of about 0.05 mg to about 1.5 mg, wherein the IN pharmaceutical formulation has a pH of about 3 to about 5, and wherein the IN pharmaceutical formulation is for IN delivery.

### Exemplary Dihydroxy Conjugate Bile Acid or Salt Thereof: Sodium Taurochenodeoxycholate (STCDC).

As an exemplary embodiment of a dihydroxy conjugate bile acid, or salt thereof, sodium taurochenodeoxycholate (STCDC) is a dihydroxy conjugate bile salt that has the molecular formula C₂₆H₄₄NNaO₆S and a molecular weight (M.W.) of 521.7 g/mol. The chemical structure of STC is shown below:

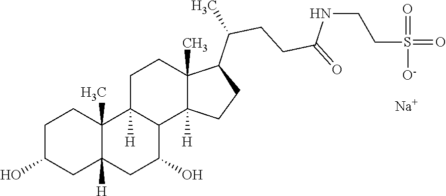

Thus, in some embodiments, the bile acid, or salt thereof, is an STCDC. In some embodiments of the IN naloxone and opioid antagonist pharmaceutical formulations, the STCDC is present at a concentration of about 1 mg/mL to about 15 mg/mL, or any concentration range subsumed therein, including but not limited to, about 1 mg/mL to about 12.5 mg/mL, about 1 mg/mL to about 10 mg/mL, about 5 mg/mL to about 11 mg/mL, about 6 mg/mL to about 13 mg/mL, about 7 mg/mL to about 12 mg/mL, about 7 mg/mL to about 9 mg/mL, about 7.5 mg/mL to about 9.5 mg/mL, about 7.5 mg/mL to about 8.5 mg/mL, about 7 mg/mL to about 9 mg/mL, or about 7 mg/mL to about 8 mg/mL. In still other embodiments of the IN naloxone and opioid antagonist pharmaceutical formulations, the STCDC is present at a concentration of about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL, about 10.5 mg/mL, about 11 mg/mL, about 11.5 mg/mL, about 12 mg/mL, about 12.5 mg/mL, about 13 mg/mL, about 13.5 mg/mL, about 14 mg/mL, about 14.5 mg/mL, or about 15 mg/mL.

In a preferred embodiment of the IN naloxone and opioid antagonist pharmaceutical formulations, the bile acid, or salt thereof, is STCDC present at a concentration of about 4 mg/mL to about 12 mg/mL.

In some embodiments of the IN naloxone and opioid antagonist pharmaceutical formulations, the STCDC, is present in a dose amount of about 0.05 mg to about 1.5 mg, or any amount range subsumed therein, including but not limited to, about 0.6 mg to about 1.3 mg, about 0.5 mg to about 1.1 mg, about 0.7 mg to about 1.2 mg, about 0.7 mg to about 0.9 mg, about 0.75 mg to about 0.95 mg, about 0.75 mg to about 0.85 mg, about 0.7 mg to about 0.9 mg, or about 0.7 mg to about 0.8 mg. In still other embodiments of IN naloxone and opioid antagonist pharmaceutical formulations, the STC is present in a dose amount of about 0.05 mg, about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, or about 1.5 mg.

In a preferred embodiment of the IN naloxone and opioid antagonist pharmaceutical formulations, the bile acid, or salt thereof, is STCDC present in a dose amount of about 0.05 mg to about 1 mg.

Thus, in some embodiments, the IN naloxone pharmaceutical formulation comprises naloxone, or a hydrate thereof, present at a concentration of about 5 mg/mL to about 50 mg/mL, an absorption enhancer comprising STCDC, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN naloxone pharmaceutical formulation consists essentially of naloxone, or a hydrate thereof, present at a concentration of about 5 mg/mL to about 50 mg/mL, an absorption enhancer comprising STCDC, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN naloxone pharmaceutical formulation consists of naloxone, or a hydrate thereof, present at a concentration of about 5 mg/mL to about 50 mg/mL, an absorption enhancer comprising STCDC, present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN naloxone pharmaceutical formulation comprises naloxone, or a hydrate thereof, present in a dose amount of about 0.5 mg to about 20 mg, an absorption enhancer comprising STCDC, present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN naloxone pharmaceutical formulation consists essentially of naloxone, or a hydrate thereof, present in a dose amount of about 0.5 mg to about 20 mg, an absorption enhancer comprising STCDC, present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN naloxone pharmaceutical formulation consists of naloxone, or a hydrate thereof, present in a dose amount of about 0.5 mg to about 20 mg, an absorption enhancer comprising STCDC, present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, buprenorphine, or a salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, buprenorphine, or a salt thereof, present in a dose amount of about 0.5 mg to about 40 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present at a concentration of about 5 mg/mL to about 50 mg/mL, buprenorphine, or a salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present at a concentration of about 5 mg/mL to about 50 mg/mL, buprenorphine, or a salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of naloxone, or a hydrate thereof, present at a concentration of about 5 mg/mL to about 50 mg/mL, buprenorphine, or a salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises naloxone, or a hydrate thereof, present in a dose amount of about 0.5 mg to about 20 mg, buprenorphine, or a salt thereof, present in a dose amount of about 0.5 mg to about 40 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of naloxone, or a hydrate thereof, present in a dose amount of about 0.5 mg to about 20 mg, buprenorphine, or a salt thereof, present in a dose amount of about 0.5 mg to about 40 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

Thus, in some embodiments, the IN pharmaceutical formulation comprises an opioid antagonist, or a hydrate or salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, an absorption enhancer comprising STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, an absorption enhancer comprising STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation consists of an opioid antagonist, or a hydrate or salt thereof, present at a concentration of about 5 mg/mL to about 50 mg/mL, buprenorphine, or a salt thereof, present at a concentration of about 5 mg/mL to about 50 mg/mL, STCDC present at a concentration of about 1 mg/mL to about 15 mg/mL, wherein the IN pharmaceutical formulation has a pH of about 3 to about 5, and wherein the IN pharmaceutical formulation is for IN delivery.

In some embodiments, the IN pharmaceutical formulation comprises an opioid antagonist, or a hydrate or salt thereof, present in a dose amount of about 0.5 mg to about 20 mg, buprenorphine, or a salt thereof, present in a dose amount of about 0.5 mg to about 40 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

In some embodiments, the IN pharmaceutical formulation consists essentially of an opioid antagonist, or a hydrate or salt thereof, present in a dose amount of about 0.5 mg to about 20 mg, buprenorphine, or a salt thereof, present in a dose amount of about 0.5 mg to about 40 mg, STCDC present in a dose amount of about 0.05 mg to about 1.5 mg, wherein the IN pharmaceutical formulation has a pH of about 3 to about 5, and wherein the IN pharmaceutical formulation is for IN delivery.

### Bile Acid or Salt Thereof Enhancement Factor (EF)

The absorption enhancement effectiveness of the bile acid or salt thereof can be quantified with reference to the following equation:

$$EF ( S ) = \frac{\overline{R} ( S )}{\overline{R} ( 0 )}$$

where EF(S) is the Bile Acid or Salt Thereof Enhancement Factor (“EF”)
R̅(S) is an average of the dose-normalized bioavailability (DN-RBA) for X (3 PK parameters: AUC_{0-30 min}, AUC_{0-180 min}, and Cₘₐₓ) by the IN route at a given bile acid or salt thereof concentration S. Here, 3 parameters are used for purposes of illustration but X can be any number of parameters, including 1 parameter or more.
R̅(0) has the same definition at S=0, by the IN route,

Additionally, the dose-normalized relative bioavailability (DN-RBA) is defined as follows:

$${R}_{X} ( S ) = \frac{{X}_{IN} ( {d}_{IN} , S ) \frac{1}{{d}_{IN}}}{{X}_{IM} ( {d}_{IM} ) \frac{1}{{d}_{IM}}}$$

where Rx is the DN-RBA for PK parameters X;
S is the concentration of bile acid or salt thereof (i.e. STC) used in the IN naloxone formulations;
X are partial AUC, AUC_{0-30 min}, and AUC_{0-180 min}, or Cₘₐₓ, note that AUC_{0-30 min}, AUC_{0-180 min}, and Cₘₐₓ are used for illustrative purposes, other PK parameters can also be assessed.
d_{IM} and d_{IN} are doses delivered by IM and IN routes, respectively.

Thus, in some embodiments of the IN naloxone and opioid antagonist formulations, the absorption enhancer comprising the bile acid, or the salt thereof, provides an EF based on an intranasal delivery (IN) v. intramuscular injection (IM) averaged pharmacokinetic (PK) results for AUC_{0-30 min}, AUC_{0-180 min}, and Cₘₐₓ, in a range of 1 to 23 or any range subsumed therein, including, but not limited to, 2 to 23, 3 to 23, 4 to 23, 5 to 23, 6 to 23, 7 to 23, 8 to 23, 9 to 23, 10 to 23, 11 to 23, 12 to 23, 13 to 23, 14 to 23, 15 to 23, 16 to 23, 17 to 23, 18 to 23, 19 to 23, 20 to 23, 21 to 23, or 22 to 23. In other embodiments of the IN naloxone and opioid antagonist formulations, the absorption enhancer comprising the bile acid, or the salt thereof, provides an EF of at least 2, 3, 4, 5, 6, 7, 8, 9, 10, 11, 12, 13, 14, 15, 16, 17, 18, 19, 20, 21, 22, or 23. In still other embodiments of the IN naloxone and opioid antagonist formulations, the absorption enhancer comprising the bile acid, or the salt thereof, provides an EF of more than 23. In some embodiments, the EF is based on the IN v. IM averaged PK results for one or more of the following PK parameters: AUC_{0-10 min}, AUC_{0-15 min}, AUC_{0-30 min}, AUC_{0-45 min}, AUC_{0-60 min}, AUC_{0-75 min}, AUC_{0-90 min}, AUC_{0-100 min}, AUC_{0-125 min}, AUC_{0-150 min}, AUC_{0-180 min}, AUC_{0-infinity}, Cₘₐₓ, Tₘₐₓ, or other suitable PK parameters.

### Naloxone Pharmaceutical Formulations Include Pharmaceutically Acceptable Excipients

In some embodiments, the IN naloxone and opioid antagonist pharmaceutical formulation includes pharmaceutically acceptable excipients. “Pharmaceutically acceptable” refers to an ingredient in the IN pharmaceutical formulation that is compatible with the other ingredients in the formulation, and does not cause excess harm to the patient receiving the IN pharmaceutical formulation.

In some embodiments, the IN naloxone and opioid antagonist pharmaceutical formulation includes a tonicity agent, such as sodium chloride, dextrose, glucose, glycerin, cellulose, mannitol, polysorbate, propylene glycol, sodium iodide, or a combination thereof, but the present disclosure is not limited thereto. In some embodiments, the IN naloxone pharmaceutical formulation includes sodium chloride as the tonicity agent.

In some embodiments, the IN naloxone and opioid antagonist pharmaceutical formulation includes the tonicity agent, such as sodium chloride, present at a concentration of about 1 mg/mL to about 10 mg/mL, or any concentration range subsumed therein, including but not limited to, about 1 mg/mL to about 9 mg/mL, about 1 mg/mL to about 8 mg/mL, about 1 mg/mL to about 7 mg/mL, about 1 mg/mL to about 6 mg/mL, about 2 mg/mL to about 9 mg/mL, about 2 mg/mL to about 8 mg/mL, about 2 mg/mL to about 7 mg/mL, about 2 mg/mL to about 6 mg/mL, about 3 mg/mL to about 9 mg/mL, about 3 mg/mL to about 8 mg/mL, about 3 mg/mL to about 7 mg/mL, about 3 mg/mL to about 6 mg/mL, about 4 mg/mL to about 9 mg/mL, about 4 mg/mL to about 8 mg/mL, about 4 mg/mL to about 7 mg/mL, about 4 mg/mL to about 6 mg/mL, about 4.5 mg/mL to about 7 mg/mL, about 4.5 mg/mL to about 6.5 mg/mL, about 5 mg/mL to about 7 mg/mL, about 5.5 mg/mL to about 6.5 mg/mL, or about 5.5 mg/mL to about 6.5 mg/mL. In other embodiments, the IN naloxone and opioid antagonist pharmaceutical formulation includes the tonicity agent, such as sodium chloride, present at a concentration of about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 5.6 mg/mL, about 5.7 mg/mL, about 5.8 mg/mL, about 5.9 mg/mL, about 6 mg/mL, about 6.1 mg/mL, about 6.2 mg/mL, about 6.3 mg/mL, about 6.4 mg/mL, about 6.5 mg/mL, about 7 mg/mL, about 7.5 mg/mL, about 8 mg/mL, about 8.5 mg/mL, about 9 mg/mL, about 9.5 mg/mL, or about 10 mg/mL.

In some embodiments, the IN naloxone and opioid antagonist pharmaceutical formulation further includes a pH adjustor, such as hydrochloric acid (HCl), sodium hydroxide (NaOH), acetic acid, ascorbic acid, sulphuric acid, tartaric acid, or a combination thereof. In other embodiments, the pH adjustor includes 10% (w/v) HCl as needed to adjust the pH to the desired pH.

In some embodiments, the IN naloxone and opioid antagonist pharmaceutical formulation comprises an additional absorption enhancer. Non-limiting examples of absorption enhancers include alcohol, aprotinin, benzalkonium chloride, benzyl alcohol, capric acid, ceramides, cetylpyridinium chloride, chitosan, cyclodextrins, deoxycholic acid, decanoyl, dimethyl sulfoxide, glyceryl monooleate, glycofurol, glycofurol, glycosylated sphingosines, glycyrrhetinic acids, 2-hydroxypropyl-β-cyclodextrin, laureth-9, lauric acid, lauroyl carnitine, lysophosphatidylcholine, menthol, poloxamer 407 or F68, poly-L-arginine, polyoxyethylene-9-lauryl ether, isopropyl myristate, isopropyl palmitate, lanolin, light mineral oil, linoleic acid, menthol, myristic acid, myristyl alcohol, oleic acid, or salt thereof, oleyl alcohol, palmitic acid, polysorbate 20, polysorbate 80, propylene glycol, polyoxyethylene alkyl ethers, polyoxylglycerides, pyrrolidone, quillaia saponin, salicylic acid, sodium salt, β-sitosterol β-D-glucoside, sodium lauryl sulfate, sucrose cocoate, taurocholic acid, taurodeoxycholic acid, taurodihydrofusidic acid, thymol, tricaprylin, triolein, and alkyl saccharides.

In some embodiments, the IN naloxone and opioid antagonist pharmaceutical formulation comprises one or more absorption enhancers selected from dodecyl maltoside, benzalkonium chloride, oleic acid, or salt thereof, polysorbate 20, polysorbate 80, sodium lauryl sulfate, and combinations thereof.

In some embodiments, the IN naloxone and opioid antagonist pharmaceutical formulation comprises an absorption enhancer present at a concentration of from about 0.001 mg/mL to about 2.5 mg/mL, from about 0.01 mg/mL to about 2.5 mg/mL, from about 0.1 mg/mL to about 2.5 mg/mL, from about 1 mg/mL to about 2.5 mg/mL, from about 0.001 mg/mL to about 1 mg/mL, from about 0.01 mg/mL to about 1 mg/mL, from about 0.1 mg/mL to about 1 mg/mL, from about 0.001 mg/mL to about 0.1 mg/mL, from about 0.01 mg/mL to about 0.1 mg/mL, or from about 0.001 mg/mL to about 0.01 mg/mL.

In some embodiments, the IN naloxone and opioid antagonist pharmaceutical formulation comprises an absorption enhancer present at a concentration of about 0.001 mg/mL, about 0.01 mg/mL, about 0.1 mg/mL, about 1 mg/mL, or about 2.5 mg/mL.

In some embodiments, the IN naloxone and opioid antagonist pharmaceutical formulation comprises a preservative. Non-limiting examples of preservatives include benzalkonium chloride, cyclodextrins, fusidic acid derivatives, phosphatidylcholines, and microspheres and liposomes.

In some embodiments, the IN naloxone and opioid antagonist pharmaceutical formulation comprises a preservative present at a concentration of from about 0.001 mg/mL to about 2.5 mg/mL, from about 0.01 mg/mL to about 2.5 mg/mL, from about 0.1 mg/mL to about 2.5 mg/mL, from about 1 mg/mL to about 2.5 mg/mL, from about 0.001 mg/mL to about 1 mg/mL, from about 0.01 mg/mL to about 1 mg/mL, from about 0.1 mg/mL to about 1 mg/mL, from about 0.001 mg/mL to about 0.1 mg/mL, from about 0.01 mg/mL to about 0.1 mg/mL, or from about 0.001 mg/mL to about 0.01 mg/mL.

In some embodiments, the IN naloxone and opioid antagonist pharmaceutical formulation comprises a preservative present at a concentration of about 0.001 mg/mL, about 0.01 mg/mL, about 0.1 mg/mL, about 1 mg/mL, or about 2.5 mg/mL.

In some embodiments, the IN naloxone and opioid antagonist pharmaceutical formulation comprises a stabilizer. In some embodiments, the stabilizer is ethylenediaminetetraacetic acid (EDTA) or a pharmaceutically acceptable salt thereof. In some embodiments, the stabilizer is disodium EDTA. In some embodiments, the IN pharmaceutical formulation comprises disodium EDTA present at a concentration of from about 0.001 mg/mL to about 1 mg/mL.

In some embodiments, the IN naloxone and opioid antagonist pharmaceutical formulation comprises an antioxidant. Non-limiting examples of antioxidants include alpha tocopherol, arachidonic acid, ascorbic acid, ascorbyl palmitate, benzethonium chloride, benzethonium bromide, benzalkonium chloride, butylated hydroxyanisole (BHA), butylated hydroxytoluene (BHT), capric acid, caproic acid, carbon dioxide, cetylpyridium chloride, chelating agents, chitosan derivatives, citric acid monohydrate, dodecyl dimethyl aminopropionate, enanthic acid, erythorbic acid, ethyl oleate, fumaric acid, glycerol oleate, glyceryl monostearate, lauric acid, limonene, linolenic acid, lysine, malic acid, menthol, methionine, monothioglycerol, myristic acid, oleic acid, palmitic acid, pelargonic acid, peppermint oil, phosphoric acid, polysorbates, potassium metabisulfite, propionic acid, propyl gallate, sodium ascorbate, sodium bisulfite, sodium caprate, sodium desoxycholate, sodium deoxyglycolate, sodium formaldehyde sulfoxylate, sodium glycocholate, sodium hydroxybenzoyal amino caprylate, sodium lauryl sulfate, sodium metabisulfite, sodium sulfite, sodium taurocholate, sodium thiosulfate, stearic acid, sulfur dioxide and combinations thereof.

In some embodiments, the IN naloxone and opioid antagonist pharmaceutical formulation comprises a buffering agent. Non-limiting examples of buffering agents include adipic acid, boric acid, calcium carbonate, calcium hydroxide, calcium lactate, calcium phosphate, tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodium hydroxide, sodium lactate, and triethanolamine.

In some embodiments, the IN naloxone and opioid antagonist pharmaceutical formulation comprises a mucosal delivery enhancing agent. Mucosal delivery enhancing agents include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of a compound(s) (e.g., biologically active compound). Enhancement of mucosal delivery can occur by any of a variety of mechanisms, including, for example, by increasing the diffusion, transport, persistence or stability of the compound, increasing membrane fluidity, modulating the availability or action of calcium and other ions that regulate intracellular or paracellular permeation, solubilizing mucosal membrane components (e.g., lipids), changing non-protein and protein sulfhydryl levels in mucosal tissues, increasing water flux across the mucosal surface, modulating epithelial junction physiology, reducing the viscosity of mucus overlying the mucosal epithelium, reducing mucociliary clearance rates, and other mechanisms. Non-limiting examples of mucosal delivery enhancing agents include aggregation inhibitory agents, charge-modifying agents, pH control agents, degradative enzyme inhibitory agents, mucolytic or mucus clearing agents, ciliostatic agents, membrane penetration-enhancing agents, alcohols, enamines, NO donor compounds, long-chain amphipathic molecules, small hydrophobic penetration enhancers, glycerol esters of acetoacetic acid, cyclodextrins or beta-cyclodextrin derivatives, medium-chain fatty acids, chelating agents, amino acids or salts thereof, N-acetylamino acids or salts thereof, inhibitors of fatty acid synthesis, inhibitors of cholesterol synthesis, modulatory agents of epithelial junction physiology, vasodilator agents, selective transport-enhancing agents, and stabilizing delivery vehicles, carriers, mucoadhesives, supports or complex-forming species with which the active agent is effectively combined, associated, contained, encapsulated or bound, resulting in stabilization of the active agent for enhanced nasal mucosal delivery, wherein the formulation of the active agent with the intranasal delivery-enhancing agents provides for increased bioavailability of the compound in a blood plasma of a subject.

Thus, in some embodiments, the IN naloxone pharmaceutical formulation comprises
about 30 mg/mL to about 50 mg/mL of naloxone, or a hydrate thereof;
about 4 mg/mL to about 12 mg/mL of an absorption enhancer comprising STC about 1 mg/mL to about 10 mg/mL of a tonicity agent;
a dose volume of about 0.1 mL to about 0.25 mL;
wherein the IN pharmaceutical formulation has a pH of about 3 to about 5.

In other embodiments, the IN pharmaceutical formulation comprises
about 35 mg/mL to about 50 mg/mL of naloxone, or a hydrate thereof;
about 4 mg/mL to about 12 mg/mL of an absorption enhancer comprising STC;
about 1 mg/mL to about 10 mg/mL of a tonicity agent;
a dose volume of about 0.25 mL;
pH adjustor as needed;
water;
wherein the IN pharmaceutical formulation has a pH of about 4 to about 5.

In some embodiments, the IN pharmaceutical formulation comprises
about 30 mg/mL to about 50 mg/mL of an opioid antagonist, or a hydrate thereof;
about 4 mg/mL to about 12 mg/mL of an absorption enhancer comprising STC; about 1 mg/mL to about 10 mg/mL of a tonicity agent;
a dose volume of about 0.1 mL to about 0.25 mL;
wherein the IN pharmaceutical formulation has a pH of about 3 to about 5.

In other embodiments, the IN pharmaceutical formulation comprises
about 35 mg/mL to about 50 mg/mL of an opioid antagonist, or a hydrate thereof;
about 4 mg/mL to about 12 mg/mL of an absorption enhancer comprising STC;
about 2 mg/mL to about 10 mg/mL of a tonicity agent;
a dose volume of about 0.25 mL;
pH adjustor as needed;
water;
wherein the IN pharmaceutical formulation has a pH of about 4 to about 5.

In some embodiments, the IN pharmaceutical formulation comprises
about 30 mg/mL to 50 mg/mL of an opioid antagonist, or a hydrate thereof;
about 5 mg/mL to about 50 mg/mL of buprenorphine, or a pharmaceutically acceptable salt thereof;
about 4 mg/mL to about 12 mg/mL of an absorption enhancer comprising STC; about 1 mg/mL to about 10 mg/mL of a tonicity agent;
a dose volume of about 0.1 mL to about 0.25 mL;
wherein the IN pharmaceutical formulation has a pH of about 3 to about 5.

In other embodiments, the IN pharmaceutical formulation comprises
about 35 mg/mL to about 50 mg/mL of an opioid antagonist, or a hydrate thereof;
about 5 mg/mL to about 50 mg/mL of buprenorphine, or a pharmaceutically acceptable salt thereof;
about 4 mg/mL to about 12 mg/mL of an absorption enhancer comprising STC;
about 2 mg/mL to about 10 mg/mL of a tonicity agent;
a dose volume of about 0.25 mL;
pH adjustor as needed;
water;
wherein the IN pharmaceutical formulation has a pH of about 4 to about 5.

In some embodiments, the IN naloxone and opioid antagonist pharmaceutical formulation for IN delivery consists essentially of about 30 mg/mL to about 50 mg/mL of naloxone, or a hydrate thereof; about 4 mg/mL to about 12 mg/mL of an absorption enhancer comprising STC; about 2 mg/mL to about 10 mg/mL of a tonicity agent; and a dose volume of about 0.1 mL to about 0.25 mL; wherein the IN pharmaceutical formulation has a pH of about 3 to about 5.

In some embodiments, the IN pharmaceutical formulation for IN delivery consists of about 30 mg/mL to about 50 mg/mL of naloxone, or a hydrate thereof; about 4 mg/mL to about 12 mg/mL of an absorption enhancer comprising STC; about 2 mg/mL to about 10 mg/mL of a tonicity agent; and a dose volume of about 0.1 mL to about 0.25 mL; wherein the IN pharmaceutical formulation has a pH of about 3 to about 5.

In some embodiments, the IN pharmaceutical formulation for IN delivery consists essentially of about 30 mg/mL to about 50 mg/mL of naloxone, or a hydrate thereof; about 5 mg/mL to about 50 mg/mL of buprenorphine, or a pharmaceutically acceptable salt thereof; about 4 mg/mL to about 12 mg/mL of an absorption enhancer comprising STC; about 2 mg/mL to about 10 mg/mL of a tonicity agent; and a dose volume of about 0.1 mL to about 0.25 mL; wherein the IN pharmaceutical formulation has a pH of about 3 to about 5.

In some embodiments, the IN pharmaceutical formulation for IN delivery consists of about 30 mg/mL to about 50 mg/mL of naloxone, or a hydrate thereof; about 5 mg/mL to about 50 mg/mL of buprenorphine, or a pharmaceutically acceptable salt thereof; about 4 mg/mL to about 12 mg/mL of an absorption enhancer comprising STC; about 2 mg/mL to about 10 mg/mL of a tonicity agent; and a dose volume of about 0.1 mL to about 0.25 mL; wherein the IN pharmaceutical formulation has a pH of about 3 to about 5.

### Methods of Use

The present disclosure also provides a method for treating opioid overdose, the method comprising administration of from about 10 μL to about 300 μL spray of an IN pharmaceutical formulation into a nostril of a subject in need thereof, wherein the IN pharmaceutical formulation comprises from about 30 mg/mL to about 50 mg/mL of an opioid antagonist, or a hydrate thereof; from about 4 mg/mL to about 12 mg/mL of an absorption enhancer comprising a bile acid, or a salt thereof; and from about 1 mg/mL to about 10 mg/mL of a tonicity agent; and wherein the pharmaceutical formulation has a pH of about 3 to about 5. In some embodiments, the opioid antagonist is naloxone. In some embodiments, the opioid antagonist is selected from the group consisting of naloxone, naltrexone, nalmefene, diprenorphine, nalorphine, nalorphine dinicotinate, levallorphan, samidorphan, nalodeine, hydrates thereof, and pharmaceutically acceptable salts thereof. In some embodiments, the IN pharmaceutical formulation comprises a second active agent, wherein the second active agent is buprenorphine, methadone, or a pharmaceutically acceptable salt thereof.

In some embodiments, the subject is an opioid overdose patient or a suspected opioid overdose patient.

In some embodiments, the subject exhibits one or more symptoms, such as respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing, erratic or stopped pulse, choking or gurgling sounds, blue or purple fingernails or lips, slack or limp muscle tone, contracted pupils, or vomiting.

In some embodiments, the symptoms may be caused by the illicit use of opioids, or by an accidental misuse of opioids.

In some embodiments, the symptoms are caused by use of an opioid. Non-limiting examples of opioids include codeine, morphine, methadone, fentanyl, oxycodone HCl, hydrocodone bitartrate, hydromorphone, oxymorphone, meperidine, propoxyphene, opium, heroin, tramadol, and tapentadol.

In some embodiments, the subject is in a lying, supine, or recovery position. In some embodiments, said patient is in a lying position. In some embodiments, the subject is in a supine position. In some embodiments, the subject is in a recovery position.

In some embodiments, the patient is not breathing.

In some embodiments, the subject is free from respiratory depression for at least about 1 hour following treatment. In some embodiments, the subject is free from respiratory depression for at least about 2 hours, about 3 hours, about 4 hours, about 5 hours, or about 6 hours following treatment.

In some embodiments, administration of the IN pharmaceutical formulation to a subject results in measurable pharmacokinetics. As used herein, Cₘₐₓ refers to the peak serum concentration of the active agent in a subject after administration. In some embodiments, administration of the IN pharmaceutical formulation results in a Cₘₐₓ of from about 300 ng/mL to about 500 ng/mL.

In some embodiments, administration of the IN pharmaceutical formulation results in a Cₘₐₓ of from about 100 ng/mL to about 150 ng/mL, from about 100 ng/mL to about 200 ng/mL, from about 100 ng/mL to about 250 ng/mL, from about 100 ng/mL to about 300 ng/mL, from about 100 ng/mL to about 350 ng/mL, from about 100 ng/mL to about 400 ng/mL, from about 100 ng/mL to about 450 ng/mL, from about 100 ng/mL to about 500 ng/mL, from about 100 ng/mL to about 550 ng/mL, from about 100 ng/mL to about 600 ng/mL, from about 100 ng/mL to about 650 ng/mL, from about 150 ng/mL to about 200 ng/mL, from about 150 ng/mL to about 250 ng/mL, from about 150 ng/mL to about 300 ng/mL, from about 150 ng/mL to about 350 ng/mL, from about 150 ng/mL to about 400 ng/mL, from about 150 ng/mL to about 450 ng/mL, from about 150 ng/mL to about 500 ng/mL, from about 150 ng/mL to about 550 ng/mL, from about 150 ng/mL to about 600 ng/mL, from about 150 ng/mL to about 650 ng/mL, from about 200 ng/mL to about 250 ng/mL, from about 200 ng/mL to about 300 ng/mL, from about 200 ng/mL to about 350 ng/mL, from about 200 ng/mL to about 400 ng/mL, from about 200 ng/mL to about 450 ng/mL, from about 200 ng/mL to about 500 ng/mL, from about 200 ng/mL to about 550 ng/mL, from about 200 ng/mL to about 600 ng/mL, from about 200 ng/mL to about 650 ng/mL, from about 250 ng/mL to about 300 ng/mL, from about 250 ng/mL to about 350 ng/mL, from about 250 ng/mL to about 400 ng/mL, from about 250 ng/mL to about 450 ng/mL, from about 250 ng/mL to about 500 ng/mL, from about 250 ng/mL to about 550 ng/mL, from about 250 ng/mL to about 600 ng/mL, from about 250 ng/mL to about 650 ng/mL, from about 300 ng/mL to about 350 ng/mL, from about 300 ng/mL to about 400 ng/mL, from about 300 ng/mL to about 450 ng/mL, from about 300 ng/mL to about 500 ng/mL, from about 300 ng/mL to about 550 ng/mL, from about 300 ng/mL to about 600 ng/mL, from about 300 ng/mL to about 650 ng/mL, from about 350 ng/mL to about 400 ng/mL, from about 350 ng/mL to about 450 ng/mL, from about 350 ng/mL to about 500 ng/mL, from about 350 ng/mL to about 550 ng/mL, from about 350 ng/mL to about 600 ng/mL, from about 350 ng/mL to about 650 ng/mL, from about 400 ng/mL to about 450 ng/mL, from about 400 ng/mL to about 500 ng/mL, from about 400 ng/mL to about 550 ng/mL, from about 400 ng/mL to about 600 ng/mL, from about 400 ng/mL to about 650 ng/mL, from about 450 ng/mL to about 500 ng/mL, from about 450 ng/mL to about 550 ng/mL, from about 450 ng/mL to about 600 ng/mL, from about 450 ng/mL to about 650 ng/mL, from about 500 ng/mL to about 550 ng/mL, from about 500 ng/mL to about 600 ng/mL, from about 500 ng/mL to about 650 ng/mL, from about 550 ng/mL to about 600 ng/mL, from about 550 ng/mL to about 650 ng/mL, or from about 600 ng/mL to about 650 ng/mL. In some embodiments, administration of the IN pharmaceutical formulation results in a Cₘₐₓ of about 100 ng/mL, about 150 ng/mL, about 200 ng/mL, about 250 ng/mL, about 300 ng/mL, about 350 ng/mL, about 400 ng/mL, about 450 ng/mL, about 500 ng/mL, about 550 ng/mL, about 600 ng/mL, or about 650 ng/mL.

In some embodiments, the Cₘₐₓ is at least 200 ng/mL or at least 400 ng/mL. In some embodiments, administration of the IN pharmaceutical formulation results in a Cₘₐₓ of least 100 ng/mL, at least 150 ng/mL, at least 250 ng/mL, at least 300 ng/mL, at least 350 ng/mL, at least 450 ng/mL, at least 500 ng/mL, at least 550 ng/mL, at least 600 ng/mL, or at least 650 ng/mL.

As used herein, Tₘₐₓ refers to the time taken to reach the maximum serum concentration (Cₘₐₓ) of the active agent in a subject after administration. In some embodiments, administration of the IN pharmaceutical formulation results in a Tₘₐₓ of from about 5 min to about 15 min.

In some embodiments, administration of the IN pharmaceutical formulation results in a Tₘₐₓ of from about 1 min to about 3 min, from about 1 min to about 5 min, from about 1 min to about 7 min, from about 1 min to about 9 min, from about 1 min to about 11 min, from about 1 min to about 13 min, from about 1 min to about 15 min, from about 1 min to about 17 min, from about 1 min to about 19 min, from about 1 min to about 21 min, from about 3 min to about 5 min, from about 3 min to about 7 min, from about 3 min to about 9 min, from about 3 min to about 11 min, from about 3 min to about 13 min, from about 3 min to about 15 min, from about 3 min to about 17 min, from about 3 min to about 19 min, from about 3 min to about 21 min, from about 5 min to about 7 min, from about 5 min to about 9 min, from about 5 min to about 11 min, from about 5 min to about 13 min, from about 5 min to about 15 min, from about 5 min to about 17 min, from about 5 min to about 19 min, from about 5 min to about 21 min, from about 7 min to about 9 min, from about 7 min to about 11 min, from about 7 min to about 13 min, from about 7 min to about 15 min, from about 7 min to about 17 min, from about 7 min to about 19 min, from about 7 min to about 21 min, from about 9 min to about 11 min, from about 9 min to about 13 min, from about 9 min to about 15 min, from about 9 min to about 17 min, from about 9 min to about 19 min, from about 9 min to about 21 min, from about 11 min to about 13 min, from about 11 min to about 15 min, from about 11 min to about 17 min, from about 11 min to about 19 min, from about 11 min to about 21 min, from about 13 min to about 15 min, from about 13 min to about 17 min, from about 13 min to about 19 min, from about 13 min to about 21 min, from about 15 min to about 17 min, from about 15 min to about 19 min, from about 15 min to about 21 min, from about 17 min to about 19 min, from about 17 min to about 21 min, or from about 17 min to about 21 min.

In some embodiments, administration of the IN pharmaceutical formulation results in a Tₘₐₓ of about 10 min. In some embodiments, administration of the IN pharmaceutical formulation results in a Tₘₐₓ of about 1 min, about 3 min, about 5 min, about 7 min, about 9 min, about 11 min, about 13 min, about 15 min, about 17 min, about 19 min, or about 21 min. In some embodiments, administration of the IN pharmaceutical formulation results in a Tₘₐₓ of less than about 15 min.

In some embodiments, administration of the IN pharmaceutical formulation results in a Tₘₐₓ of less than about 21 min, less than about 19 min, less than about 17 min, less than about 13 min, less than about 11 min, less than about 9 min, less than about 7 min, less than about 5 min, less than about 3 min, or less than about 1 min.

As used herein, AUC refers to the area under the curve, wherein the curve is a plot of concentration of active agent as a function of time after administration. AUC refers to the area under the curve from initial administration up to time x. For example, AUC_{30 min} refers to the area under the curve from initial administration up to 30 min after administration. In some embodiments, administration of the IN pharmaceutical formulation results in an AUC_{30 min} of from about 3000 ng/mL·min to about 9000 ng/mL·min.

In some embodiments, administration of the IN pharmaceutical formulation results in an AUC_{30 min} of from about 2000 ng/mL·min to about 2500 ng/mL·min, from about 2000 ng/mL·min to about 3000 ng/mL·min, from about 2000 ng/mL·min to about 3500 ng/mL·min, from about 2000 ng/mL·min to about 4000 ng/mL·min, from about 2000 ng/mL·min to about 4500 ng/mL·min, from about 2000 ng/mL·min to about 5000 ng/mL·min, from about 2000 ng/mL·min to about 5500 ng/mL·min, from about 2000 ng/mL·min to about 6000 ng/mL·min, from about 2000 ng/mL·min to about 6500 ng/mL·min, from about 2000 ng/mL·min to about 7000 ng/mL·min, from about 2000 ng/mL·min to about 7500 ng/mL·min, from about 2000 ng/mL·min to about 8000 ng/mL·min, from about 2000 ng/mL·min to about 8500 ng/mL·min, from about 2000 ng/mL·min to about 9000 ng/mL·min, from about 2500 ng/mL·min to about 3000 ng/mL·min, from about 2500 ng/mL·min to about 3500 ng/mL·min, from about 2500 ng/mL·min to about 4000 ng/mL·min, from about 2500 ng/mL·min to about 4500 ng/mL·min, from about 2500 ng/mL·min to about 5000 ng/mL·min, from about 2500 ng/mL·min to about 5500 ng/mL·min, from about 2500 ng/mL·min to about 6000 ng/mL·min, from about 2500 ng/mL·min to about 6500 ng/mL·min, from about 2500 ng/mL·min to about 7000 ng/mL·min, from about 2500 ng/mL·min to about 7500 ng/mL·min, from about 2500 ng/mL·min to about 8000 ng/mL·min, from about 2500 ng/mL·min to about 8500 ng/mL·min, from about 2500 ng/mL·min to about 9000 ng/mL·min, from about 3000 ng/mL·min to about 3500 ng/mL·min, from about 3000 ng/mL·min to about 4000 ng/mL·min, from about 3000 ng/mL·min to about 4500 ng/mL·min, from about 3000 ng/mL·min to about 5000 ng/mL·min, from about 3000 ng/mL·min to about 5500 ng/mL·min, from about 3000 ng/mL·min to about 6000 ng/mL·min, from about 3000 ng/mL·min to about 6500 ng/mL·min, from about 3000 ng/mL·min to about 7000 ng/mL·min, from about 3000 ng/mL·min to about 7500 ng/mL·min, from about 3000 ng/mL·min to about 8000 ng/mL·min, from about 3000 ng/mL·min to about 8500 ng/mL·min, from about 3000 ng/mL·min to about 9000 ng/mL·min, from about 3500 ng/mL·min to about 4000 ng/mL·min, from about 3500 ng/mL·min to about 4500 ng/mL·min, from about 3500 ng/mL·min to about 5000 ng/mL min, from about 3500 ng/mL·min to about 5500 ng/mL·min, from about 3500 ng/mL·min to about 6000 ng/mL·min, from about 3500 ng/mL·min to about 6500 ng/mL·min, from about 3500 ng/mL·min to about 7000 ng/mL·min, from about 3500 ng/mL·min to about 7500 ng/mL·min, from about 3500 ng/mL·min to about 8000 ng/mL min, from about 3500 ng/mL·min to about 8500 ng/mL·min, from about 3500 ng/mL·min to about 9000 ng/mL·min, from about 4000 ng/mL·min to about 4500 ng/mL·min, from about 4000 ng/mL·min to about 5000 ng/mL·min, from about 4000 ng/mL·min to about 5500 ng/mL·min, from about 4000 ng/mL·min to about 6000 ng/mL·min, from about 4000 ng/mL·min to about 6500 ng/mL·min, from about 4000 ng/mL·min to about 7000 ng/mL·min, from about 4000 ng/mL·min to about 7500 ng/mL·min, from about 4000 ng/mL·min to about 8000 ng/mL·min, from about 4000 ng/mL·min to about 8500 ng/mL·min, from about 4000 ng/mL·min to about 9000 ng/mL·min, from about 4500 ng/mL·min to about 5000 ng/mL·min, from about 4500 ng/mL·min to about 5500 ng/mL·min, from about 4500 ng/mL·min to about 6000 ng/mL·min, from about 4500 ng/mL·min to about 6500 ng/mL·min, from about 4500 ng/mL·min to about 7000 ng/mL·min, from about 4500 ng/mL·min to about 7500 ng/mL·min, from about 4500 ng/mL·min to about 8000 ng/mL·min, from about 4500 ng/mL·min to about 8500 ng/mL·min, from about 4500 ng/mL·min to about 9000 ng/mL·min, from about 5000 ng/mL·min to about 5500 ng/mL·min, from about 5000 ng/mL·min to about 6000 ng/mL·min, from about 5000 ng/mL·min to about 6500 ng/mL·min, from about 5000 ng/mL·min to about 7000 ng/mL·min, from about 5000 ng/mL·min to about 7500 ng/mL·min, from about 5000 ng/mL·min to about 8000 ng/mL min, from about 5000 ng/mL·min to about 8500 ng/mL·min, from about 5000 ng/mL·min to about 9000 ng/mL·min, from about 5500 ng/mL·min to about 6000 ng/mL min, from about 5500 ng/mL·min to about 6500 ng/mL·min, from about 5500 ng/mL·min to about 7000 ng/mL·min, from about 5500 ng/mL·min to about 7500 ng/mL·min, from about 5500 ng/mL·min to about 8000 ng/mL·min, from about 5500 ng/mL·min to about 8500 ng/mL·min, from about 5500 ng/mL·min to about 9000 ng/mL·min, from about 6000 ng/mL·min to about 6500 ng/mL·min, from about 6000 ng/mL·min to about 7000 ng/mL·min, from about 6000 ng/mL·min to about 7500 ng/mL·min, from about 6000 ng/mL·min to about 8000 ng/mL·min, from about 6000 ng/mL·min to about 8500 ng/mL·min, from about 6000 ng/mL·min to about 9000 ng/mL·min, from about 6500 ng/mL·min to about 7000 ng/mL·min, from about 6500 ng/mL·min to about 7500 ng/mL·min, from about 6500 ng/mL·min to about 8000 ng/mL·min, from about 6500 ng/mL·min to about 8500 ng/mL·min, from about 6500 ng/mL·min to about 9000 ng/mL·min, from about 7000 ng/mL·min to about 7500 ng/mL·min, from about 7000 ng/mL·min to about 8000 ng/mL·min, from about 7000 ng/mL·min to about 8500 ng/mL·min, from about 7000 ng/mL·min to about 9000 ng/mL·min, from about 7500 ng/mL·min to about 8000 ng/mL·min, from about 7500 ng/mL·min to about 8500 ng/mL·min, from about 7500 ng/mL·min to about 9000 ng/mL·min, from about 8000 ng/mL·min to about 8500 ng/mL·min, from about 8000 ng/mL·min to about 9000 ng/mL·min, or from about 8500 ng/mL·min to about 9000 ng/mL·min.

In some embodiments, administration of the IN pharmaceutical formulation results in an AUC_{30 min} of about 6000 ng/mL·min.

In some embodiments, administration of the IN pharmaceutical formulation results in an AUC_{30 min} of about 2000 ng/mL·min, about 2500 ng/mL·min, about 3000 ng/mL·min, about 3500 ng/mL·min, about 4000 ng/mL·min, about 4500 ng/mL·min, about 5000 ng/mL·min, about 5500 ng/mL·min, about 6500 ng/mL·min, about 7000 ng/mL·min, about 7500 ng/mL·min, about 8000 ng/mL·min, about 8500 ng/mL·min, or about 9000 ng/mL·min, In some embodiments, administration of the IN pharmaceutical formulation results in an AUC_{30 min} greater than about 4500 ng/mL·min.

In some embodiments, administration of the IN pharmaceutical formulation results in an AUC_{30 min} greater than about 2000 ng/mL·min, greater than about 2500 ng/mL·min, greater than about 3000 ng/mL·min, greater than about 3500 ng/mL·min, greater than about 4000 ng/mL·min, greater than about 5000 ng/mL·min, greater than about 5500 ng/mL·min, greater than about 6000 ng/mL·min, greater than about 6500 ng/mL·min, greater than about 7000 ng/mL·min, greater than about 7500 ng/mL·min, greater than about 8000 ng/mL·min, greater than about 8500 ng/mL·min, or greater than about 9000 ng/mL·min.

As used herein, relative bioavailability (RBA) refers to the bioavailability of an active agent after IN administration relative to the bioavailability of an active agent after IM administration. In some embodiments, the RBA after administration of the IN pharmaceutical formulation is from about 30% to about 60%.

In some embodiments, the RBA after administration of the IN pharmaceutical formulation is from about 10% to about 20%, from about 10% to about 30%, from about 10% to about 40%, from about 10% to about 50%, from about 10% to about 60%, from about 10% to about 70%, from about 10% to about 80%, from about 10% to about 90%, from about 10% to about 100%, from about 20% to about 30%, from about 20% to about 40%, from about 20% to about 50%, from about 20% to about 60%, from about 20% to about 70%, from about 20% to about 80%, from about 20% to about 90%, from about 20% to about 100%, from about 30% to about 40%, from about 30% to about 50%, from about 30% to about 70%, from about 30% to about 80%, from about 30% to about 90%, from about 30% to about 100%, from about 40% to about 50%, from about 40% to about 60%, from about 40% to about 70%, from about 40% to about 80%, from about 40% to about 90%, from about 40% to about 100%, from about 50% to about 60%, from about 50% to about 70%, from about 50% to about 80%, from about 50% to about 90%, from about 50% to about 100%, from about 60% to about 70%, from about 60% to about 80%, from about 60% to about 90%, from about 60% to about 100%, from about 70% to about 80%, from about 70% to about 90%, from about 70% to about 100%, from about 80% to about 90%, from about 80% to about 100%, or from about 90% to about 100%.

In some embodiments, the RBA after administration of the IN pharmaceutical formulation is about 40%. In some embodiments, the RBA after administration of the IN pharmaceutical formulation is about 10%, about 20%, about 30%, about 50%, about 60%, about 70%, about 80%, about 90%, or about 100%. In some embodiments, the RBA after administration of the IN pharmaceutical formulation is about 1.5 fold larger than the RBA after administration of a pharmaceutical formulation that does not comprise an absorption enhancer.

In some embodiments, the RBA after administration of the IN pharmaceutical formulation is about 1.1, about 1.2, about 1.3, about 1.4, about 1.5, about 1.6, about 1.7, about 1.8, about 1.9, about 2, about 2.5, about 3, about 4, about 5, about 6, about 7, about 8, about 9, or about 10 fold larger than the RBA after administration of a pharmaceutical formulation that does not comprise an absorption enhancer.

### EXAMPLES

### Example 1

### Bile Acids and Salts Thereof (STC) as Absorption Enhancer for IN Delivery of Naloxone

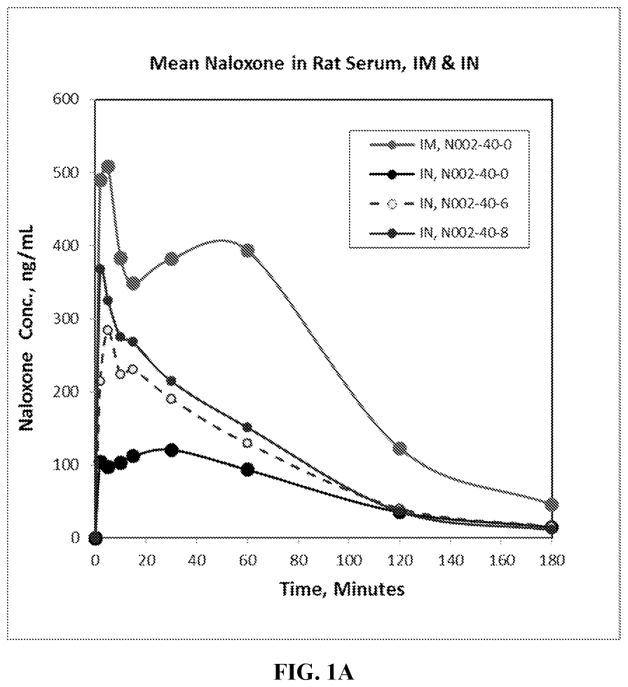

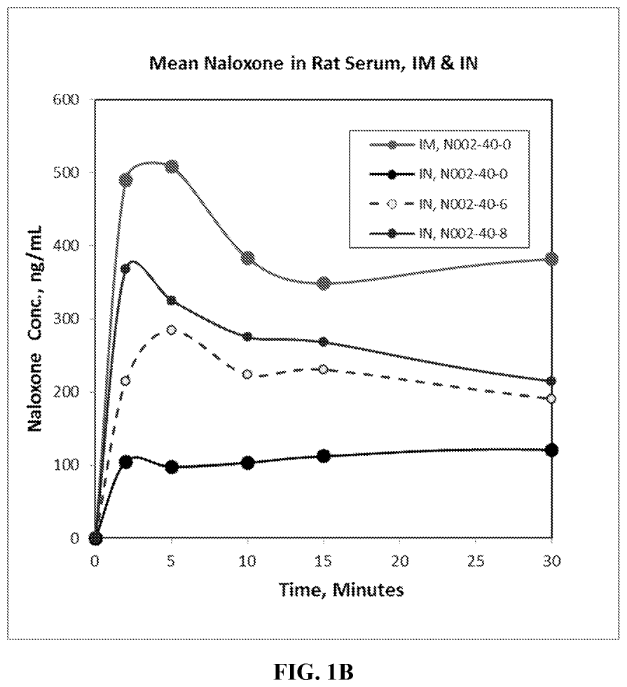

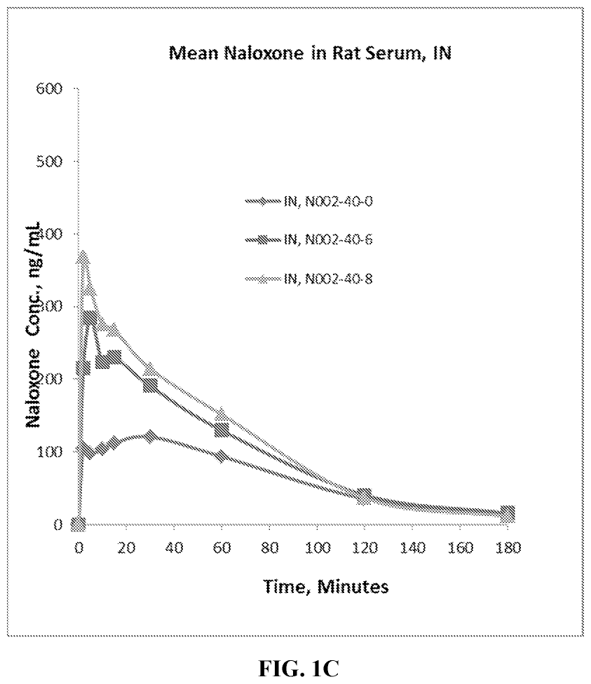

Example 1 is an animal study using sodium taurocholate (STC) as the bile acid, or salt thereof, for enhancing the absorption of a different active agent, namely naloxone. Example 1 was designed to study the relative bioavailability of naloxone in the route of intranasal administration (IN delivery) relative to administration by intramuscular injection (IM). The naloxone concentrations in rat's serum are determined at the baseline and at various post administration time points after IN delivery (FIG. 1 AB/C). The IN bioavailability is then calculated using PK data of area under the curve (AUC) and Cₘₐₓ.

In Example 1 effects of a bile acid, or salt thereof, such as STC, on naloxone IN delivery using various formulations with STC concentrations ranging from 0 mg/mL to 8 mg/mL, and naloxone HCl of about 40 mg/mL were studied. Table 1 provides the naloxone and STC formulations tested for Example 1. Note that the naloxone in the Table 1 formulations uses naloxone hydrochloride (HCl) dihydrate. Also, in Table 1, “Conc.” refers to concentration.

TABLE 1
Naloxone and STC Formulations Tested in Example 1 (Excipients not shown)

| | | Naloxone Conc. | STC Conc. | Dose | Number | |
|---|---|---|---|---|---|---|
| | Delivery | (mg/mL)/Dose | (mg/mL)/Dose | Volume | of rats | |
| No. | Route | (mg) | (mg) | (μL) | (n=) | Description |
| 1 | IM | 40/1 | 0/0 | 25 | 8 | IM, N002-40-0 |
| 2 | IN | 40/1 | 0/0 | 25 | 8 | IN, N002-40-0 |
| 3 | IN | 40/1 | 6/0.15 | 25 | 8 | IN, N002-40-6 |
| 4 | IN | 40/1 | 8/0.20 | 25 | 8 | IN, N002-40-8 |

In Table 1, Formulations Nos. 1-2 also included about 2.75 mg/mL of sodium chloride as a tonicity agent, pH adjustor as needed (10% HCl), pH 4.2, and water (q.s.). Formulations Nos. 3-4 also included about 2.0 mg/mL of sodium chloride as a tonicity agent, pH adjustor as needed (10% HCl), pH 4.5, and water (q.s.).

Animal Study design: Thirty two (32) rats (Male:Female=1:1) were randomly divided into 4 arms (M, T1-T3), corresponding to formulation Nos. 1-4 as listed in Table 1; each arm had a total of 8 rats. Individual rats were assigned a sequential ID number at the time of receipt. At the time of treatment group assignment, rat ID numbers were randomly assigned to each treatment group. The rats in each arm received one particular test article as listed in Table 1. The test article was intramuscularly (IM) injected into rats in arm M (No. 1) while for T1-T3 (Nos. 2-4), the route of administration was intranasal (IN).

For IN delivery of Formulations Nos. 2-4 of Table 1, each formulation was delivered by IN in the amount of 25 μL to the right nostril of the rat using a 27G Blunt Needle (Instech, Part No. LS27) connected to a glass syringe (Hamilton, Model 1725, 250 μL). The rat was anesthetized to effect (isoflurane, 5% for approximately 5 minutes) before administration.

For the IM administration of Formulation No. 1 of Table 1, this formulation was intramuscularly injected in the amount of 25 μL to the right back thigh using a 31G insulin syringe (BD Insulin Syringe, 0.3 mL, ½ unit). To be consistent with the IN administration, the rats that received IM injection were also anesthetized (isoflurane, 5%) for 5 minutes before injection.

Tables 2 and 3 provide a summary of the pharmacokinetic (PK) results for Example 1. In Table 3, the IM Formulation No. 1 from Table 1 was used as the reference IM for determining the relative bioavailabilities (RBA) compared to the IN Formulations Nos. 2-4. Additionally, FIGS. 1A-1C depict some of the key PK results provided in Tables 2 and 3. In particular, FIG. 1A depicts the mean naloxone concentration in rat serum from 0 min. to 180 mins. FIG. 1B depicts the mean naloxone concentration in rat serum from 0 min. to 30 mins. FIG. 1C depicts the mean naloxone concentration in rat serum by IN delivery.

TABLE 2
Summary of PK Results for Example 1.

| | | | | | | Active | Active |
|---|---|---|---|---|---|---|---|
| Formulation | | | AUC, ng/mL · min | | | Agent | Agent |
| No. (From | Cₘₐₓ | Tₘₐₓ, | AUC_{0-30 min.} ± | AUC_{0-60 min.} ± | AUC_{0-180 min.} ± | Dose | Dose |
| Table 1) | (ng/mL) | min | S.D. | S.D. | S.D. | (mg/kg) | (mg/m²) |
| 1 | 667 | 18 | 11526 ± 4146 | 23163 ± 5980 | 43726 ± 8257 | 3.6 | 24 |
| 2 | 162 | 18 | 3192 ± 1008 | 6409 ± 1592 | 11786 ± 3320 | 3.5 | 24 |
| 3 | 310 | 8.8 | 6528 ± 1721 | 11335 ± 2884 | 18118 ± 4797 | 3.5 | 23 |
| 4 | 414 | 6.6 | 7891 ± 2974 | 13382 ± 4823 | 20434 ± 6781 | 3.4 | 23 |

TABLE 3
Relative Bioavailability (RBA), IN vs. IM for Example 1.

| | Relative Bioavailability (RBA). IN vs. IM | | | | |
|---|---|---|---|---|---|
| Formulation | | | | Mean AUC ₍₀₋ | |
| No. (From | AUC₀₋ | AUC₀₋ | AUC₀₋ | _{30 min, 0-60 min,} | |
| Table 1) | _{30 min} | ₆₀ₘᵢₙ | _{180 min} | _{0-180 min.)} | Cₘₐₓ |
| 2 | 28% | 28% | 27% | 27% | 24% |
| 3 | 57% | 49% | 41% | 49% | 46% |
| 4 | 68% | 58% | 47% | 58% | 62% |

Notably, as shown in Table 3, the mean relative bioavailability (RBA) of naloxone with no STC (Formulation No. 2 in Table 1) in IN delivery in rats is 27% relative to the IM administration route (Formulation No. 1 in Table 1). If the IN naloxone formulation contains 6 mg/mL of STC (Formulation No. 3 in Table 1), then the mean RBA is significantly increased to 49%, a factor of 1.8 times increase, compared to naloxone with no STC (Formulation No. 2 in Table 1). Moreover, if the IN naloxone formulation contains 8 mg/mL of STC (Formulation No. 4 in Table 1), then the mean RBA is significantly increased to 58%, a factor of 2.1 times increase, compared to naloxone with no STC (Formulation No. 2 in Table 1).

Also notable, the RBA for Cₘₐₓ also demonstrates similar enhancement effects by the bile acid, or salt thereof, STC. In particular, as shown in Table 3, in Formulation No. 2, which does not contain STC, the naloxone RBA Cₘₐₓ is only 24%. When the formulation contains 6 mg/mL of STC such as in Formulation No. 3, the RBA Cₘₐₓ is increased to 46%, a factor of 1.8 times increase. Further, in the formulation containing 6 mg/mL of STC such as Formulation No. 4, the RBA Cₘₐₓ is 58%, a factor of 2.1 times increase compared to the no STC formulation.

Also notable, in Table 2, the time of Tₘₐₓ in IN delivery is shorter when 6 mg/mL or 8 mg/mL STC is included in the naloxone formulation, compared to no STC in the formulation.

Combined information in Table 2 and Table 3 demonstrated that:

(1) The RBA of IN delivery increases from less than 30% to about 60% as STC concentration increases in the formulation, indicating a STC-mediated absorption enhancement effect.

(2) Intranasal delivery with STC could reach to a comparable level of drug absorption (Cₘₐₓ and AUC) with that of standard IM route of administration.

(3) Intranasal delivery with STC can lead to decreased Tₘₐₓ compared with that of IM, suggestive of quicker onset of drug action when required.

### Example 2

Example 2 provides exemplary formulations for IM or IN delivery comprising an opioid antagonist and pharmaceutically acceptable excipients (Table 4).

TABLE 4
Exemplary opioid antagonist formulations

| | | Opioid | | |
|---|---|---|---|---|
| | | antagonist | STC | |
| | | Conc. | Conc. | Dose |
| | Delivery | (mg/mL)/ | (mg/mL)/ | Volume |
| No. | Route | Dose (mg) | Dose (mg) | (μL) |
| 1 | IM | 40/1 | 0/0 | 25 |
| 2 | IN | 40/1 | 0/0 | 25 |
| 3 | IN | 40/1 | 6/0.15 | 25 |
| 4 | IN | 40/1 | 8/0.20 | 25 |

In Table 4, all formulations also included a tonicity agent (NaCl), and a pH adjustor as needed (10% HCl), pH 3-5, and water (q.s.).

### Example 3

Example 3 provides exemplary IN formulations for IN delivery comprising an opioid antagonist and pharmaceutically acceptable excipients (Table 5).

TABLE 5
Exemplary opioid antagonist formulations

| | No. 1 | | No. 2 | | No. 3 | | No. 4 | | No. 5 | | No. 6 | |
|---|---|---|---|---|---|---|---|---|---|---|---|---|
| Opioid antagonist | 40 | mg/mL | 30-50 | mg/mL | 30-50 | mg/mL | 35 | mg/mL | 30-40 | mg/mL | 40-50 | mg/mL |
| STC | 5 | mg/mL | 1-15 | mg/mL | 1-15 | mg/mL | 4 | mg/mL | 1-10 | mg/mL | 5-15 | mg/mL |

| | | | | | | | | | |
|---|---|---|---|---|---|---|---|---|---|
| Benzalkonium | — | — | — | 0.01 | mg/mL | 0.001-0.1 | mg/mL | 0.001-0.1 | mg/mL |
| chloride | | | | | | | | | |

| | | | | | | | | | |
|---|---|---|---|---|---|---|---|---|---|
| Dodecyl maltoside | 0.01 | mg/mL | 0.001-0.1 | mg/mL | 0.001-0.1 | mg/mL | — | — | — |

| | | | | | | |
|---|---|---|---|---|---|---|
| pH | 4.5 | 4 | 6 | 4.5 | 5 | 4 |

In Table 5, all formulations also include about 2.75 mg/mL of sodium chloride as a tonicity agent, pH adjustor as needed (10% HCl), and water (q.s.).

### Example 4

Example 4 provides exemplary IN combination formulations for IN delivery comprising naloxone or a hydrate thereof, buprenorphine, and pharmaceutically acceptable excipients (Table 6).

TABLE 6
Exemplary naloxone/buprenorphine combination formulations

| | No. 1 | | No. 2 | | No. 3 | | No. 4 | | No. 5 | | No. 6 | |
|---|---|---|---|---|---|---|---|---|---|---|---|---|
| Naloxone (or a | 40 | mg/mL | 30-50 | mg/mL | 30-50 | mg/mL | 35 | mg/mL | 30-40 | mg/mL | 40-50 | mg/mL |
| hydrate thereof) | | | | | | | | | | | | |
| Buprenorphine | 10 | mg/mL | 5-50 | mg/mL | 10-60 | mg/mL | 7 | mg/mL | 5-30 | mg/mL | 30-60 | mg/mL |
| STC | 5 | mg/mL | 1-15 | mg/mL | 1-15 | mg/mL | 4 | mg/mL | 1-10 | mg/mL | 5-15 | mg/mL |

| | | | | | | | | | |
|---|---|---|---|---|---|---|---|---|---|
| Benzalkonium | — | — | — | 0.01 | mg/mL | 0.001-0.1 | mg/mL | 0.001-0.1 | mg/mL |
| chloride | | | | | | | | | |

| | | | | | | | | | |
|---|---|---|---|---|---|---|---|---|---|
| Dodecyl maltoside | 0.01 | mg/mL | 0.001-0.1 | mg/mL | 0.001-0.1 | mg/mL | — | — | — |

| | | | | | | |
|---|---|---|---|---|---|---|
| pH | 4.5 | 4 | 6 | 4.5 | 5 | 4 |

In Table 6, all formulations also include about 2.75 mg/mL of sodium chloride as a tonicity agent, pH adjustor as needed (10% HCl), and water (q.s.).

### Example 5

Example 5 provides exemplary IN combination formulations for IN delivery comprising an opioid antagonist, buprenorphine, and pharmaceutically acceptable excipients (Table 7).

TABLE 7
Exemplary opioid antagonist/buprenorphine combination formulations

| | No. 1 | | No. 2 | | No. 3 | | No. 4 | | No. 5 | | No. 6 | |
|---|---|---|---|---|---|---|---|---|---|---|---|---|
| Opioid antagonist | 40 | mg/mL | 30-50 | mg/mL | 30-50 | mg/mL | 35 | mg/mL | 30-40 | mg/mL | 40-50 | mg/mL |
| Buprenorphine | 10 | mg/mL | 5-50 | mg/mL | 10-60 | mg/mL | 7 | mg/mL | 5-30 | mg/mL | 30-60 | mg/mL |
| STC | 5 | mg/mL | 1-15 | mg/mL | 1-15 | mg/mL | 4 | mg/mL | 1-10 | mg/mL | 5-15 | mg/mL |

| | | | | | | | | | |
|---|---|---|---|---|---|---|---|---|---|
| Benzalkonium | — | — | — | 0.01 | mg/mL | 0.001-0.1 | mg/mL | 0.001-0.1 | mg/mL |
| chloride | | | | | | | | | |

| | | | | | | | | | |
|---|---|---|---|---|---|---|---|---|---|
| Dodecyl maltoside | 0.01 | mg/mL | 0.001-0.1 | mg/mL | 0.001-0.1 | mg/mL | — | — | — |

| | | | | | | |
|---|---|---|---|---|---|---|
| pH | 4.5 | 4 | 6 | 4.5 | 5 | 4 |

In Table 7, all formulations include sodium chloride as a tonicity agent, and a pH adjustor as needed.

What is claimed:

1. An intranasal (IN) pharmaceutical formulation comprising: naloxone, or a hydrate thereof an absorption enhancer comprising a bile acid, or a salt thereof; wherein the formulation has a pH of about 3 to about 5.

2. The IN pharmaceutical formulation of claim 1, wherein the naloxone, or the hydrate thereof, is present at a concentration of about 5 mg/mL to about 50 mg/mL.

3. The IN pharmaceutical formulation of claim 1, wherein the naloxone hydrate is naloxone HCl dihydrate.

4. The IN pharmaceutical formulation of claim 1, wherein the naloxone is naloxone HCl anhydrous.

5. The IN pharmaceutical formulation of claim 3, wherein the naloxone HCl dihydrate is present in a dose amount of about 0.50 mg to about 20 mg.

6. The IN pharmaceutical formulation of claim 4, wherein the naloxone HCl anhydrous is present in a dose amount of about 0.50 mg to about 20 mg.

7. The IN pharmaceutical formulation of claim 1, wherein the bile acid, or the salt thereof, is present at a concentration of about 1 mg/mL to about 15 mg/mL.

8. The IN pharmaceutical formulation of claim 1, wherein the bile acid, or the salt thereof, is present in a dose amount of about 0.1 mg to about 1.5 mg.

9. The IN pharmaceutical formulation of claim 1, wherein the bile acid, or the salt thereof, is a trihydroxy conjugate.

10. The IN pharmaceutical formulation of any one of claims 1 to 9, wherein the bile acid, or salt thereof, is a trihydroxy conjugate selected from the group consisting of glycocholate (GC), taurocholate (TC), glycohyocholate (GHC), taurohyocholate (THC), tauro-α-muricholate (T-α-MC), tauro-β-muricholate (T-β-MC), and combinations thereof.

11. The IN pharmaceutical formulation of claim 1, wherein the bile acid, or salt thereof, is a trihydroxy conjugate selected from the group consisting of sodium glycocholate (SGC), sodium taurocholate (STC), sodium glycohyocholate (SGHC), sodium taurohyocholate (STHC), sodium tauro-α-muricholate (S-T-α-MC), sodium tauro-β-muricholate (S-T-β-MC), and combinations thereof.

12. The IN pharmaceutical formulation of claim 1, wherein the bile acid, or the salt thereof, is a dihydroxy conjugate.

13. The IN pharmaceutical formulation of claim 1, wherein the bile acid, or salt thereof, is a dihydroxy conjugate selected from the group consisting of sodium tauroursodeoxycholate (STUDC), sodium taurohyodeoxycholate (STUDC), sodium glycohyodeoxycholate (SGUDC), sodium glycochenodeoxycholate (SGCDC), taurodeoxycholate (TDC), sodium taurodeoxycholate (STDC), sodium taurochenodeoxycholate (STCDC), sodium glycodeoxychoate (SGDC), sodium glycoursodeoxycholate (SGUDC), and combinations thereof.

14. The IN pharmaceutical formulation of claim 1, wherein the bile, or the salt thereof, is a dihydroxy conjugate selected from the group consisting of tauroursodeoxycholate (TUDC), taurohyodeoxycholate (THDC), glycohyodeoxycholate (GHDC), glycochenodeoxycholate (GCDC), taurodeoxycholate (TDC), taurochenodeoxycholate (TCDC), glycodeoxychoate (GDC), glycoursodeoxycholate (GUDC), and combinations thereof.

15. The IN pharmaceutical formulation of claim 1, wherein the bile acid, or the salt thereof, is an unconjugated form.

16. The IN pharmaceutical formulation of claim 1, wherein the bile acid, or the salt thereof, is an unconjugated form selected from the group consisting of cholate, deoxycholate (DC), chenodeoxycholate (CDC), and combinations thereof.

17. The IN pharmaceutical formulation of claim 1, wherein the bile acid, or the salt thereof, is an unconjugated form selected from the group consisting of sodium cholate (SC), sodium deoxycholate (SDC), sodium chenodeoxycholate (SCDC), and combinations thereof.

18. The IN pharmaceutical formulation of claim 1, wherein the bile acid, or the salt thereof, is STC.

19. The IN pharmaceutical formulation of claim 18, wherein the STC is present at a concentration of about 1 mg/mL to about 15 mg/mL.

20. The IN pharmaceutical formulation of claim 1, wherein the bile acid, or the salt thereof, is STCDC.

21.-153. (canceled)
